(19) United States
(12) Patent Application Publication
(10) Pub. No.: US 2011/0275065 A1
(43) Pub. Date: Nov. 10, 2011

(54) METHODS AND COMPOSITIONS FOR THE DIAGNOSIS AND TREATMENT OF THYROID CANCER

(75) Inventors: Paul Walfish, Toronto (CA); Ranju Ralhan, Thornhill (CA); K. W. Michael Siu, Toronto (CA)

(73) Assignees: Ranju Ralhan, Thomhill (CA); Paul Walfish, Toronto (CA)

(21) Appl. No.: 13/102,638

(22) Filed: May 6, 2011

Related U.S. Application Data
(60) Provisional application No. 61/332,381, filed on May 7, 2010.

(51) Int. Cl. C12Q 1/68; G01N 33/48; G01N 33/566

(52) U.S. Cl. 435/6.1; 436/501; 436/64

(57) ABSTRACT

Methods for detecting thyroid cancer or thyroid cancer status in a subject are described comprising measuring novel markers or polynucleotides encoding the markers in a sample from the subject. The invention also provides localization or imaging methods for thyroid cancer, and kits for carrying out the methods of the invention. The invention also contemplates therapeutic applications for thyroid cancer employing the novel markers, polynucleotides encoding the markers, and/or binding agents for the markers.

### CROSS REFERENCE TO RELATED APPLICATION

This application claims the benefit under 35 USC §119(e) of U.S. Provisional Patent Application No. 61/332,381, filed May 7, 2010. This application is incorporated herein in its entirety.

### FIELD OF THE INVENTION

The invention relates to compositions, kits, and methods for detecting, screening for, diagnosing, monitoring, and characterizing thyroid cancer.

### BACKGROUND OF THE INVENTION

Thyroid cancers are the most common malignancy of the endocrine system. [1] There is currently a lack of molecular markers to predict the aggressiveness of thyroid cancers. Currently fine-needle aspiration (FNA) is the most accurate preoperative technique for diagnosis of thyroid nodules. However, even when using ultrasound-guided FNA, inconclusive biopsy results are quite common (10-20% of all cases). [2] These patients normally undergo subsequent surgery to remove their thyroid gland—an invasive procedure that is usually unnecessary as the majority of the suspected lesions turn out to be benign (>80%). [3] Patients with inconclusive results and malignant tumors are also at risk for not undergoing adequate treatment as many of them undergo an initial thyroid lobectomy that must be followed up with another surgery to complete the thyroidectomy following diagnosis. [3] Additionally, an issue also arises due to the fact that while most papillary thyroid cancers are non-aggressive with limited to no metastasis, a small percentage are in-fact aggressive and may produce distant metastasis leading to higher mortality. [1] This establishes an urgent need for identifying biomarkers to distinguish benign thyroid nodules from malignant and aggressive carcinomas.

### SUMMARY OF THE INVENTION

The present invention relates to markers of thyroid cancer. In particular, polypeptides and domains thereof disclosed in Table 1 (collectively referred to herein as “Polypeptide Thyroid Cancer Markers”), and polynucleotides encoding such polypeptides and domains thereof (collectively referred to herein as “Polynucleotide Thyroid Cancer Markers”) constitute biomarkers for thyroid cancer. Polypeptide Thyroid Cancer Markers and Polynucleotide Thyroid Cancer Markers, and portions or fragments thereof, are sometimes collectively referred to herein as “Thyroid Cancer Markers”.

Thus, Thyroid Cancer Markers and agents that interact with the Thyroid Cancer Markers, may be used in detecting, screening for, diagnosing, characterizing, and monitoring thyroid cancer (i.e., monitoring progression of the cancer or the effectiveness of a therapeutic treatment) or for assisting same, in the identification of subjects with a predisposition to thyroid cancer, and in determining patient survival. In aspects of the invention, the Thyroid Cancer Markers are used in characterizing the aggressiveness of a thyroid cancer. In some aspects of the invention, the Thyroid Cancer Markers are used to determine metastatic potential or patient survival.

A method of the invention wherein Thyroid Cancer Marker(s) are assayed can have enhanced sensitivity and/or specificity relative to a method assaying other markers. The enhanced clinical sensitivity may be about a 5-10% increase, in particular 6-9% increase, more particularly 8% increase in sensitivity. In an embodiment, a method of the invention where one or more Thyroid Cancer Marker(s) detected in tumor samples provides a thyroid cancer clinical sensitivity of at least about 80 to 99%, in particular 90 to 95%, more particularly 91%, 92%, 93%, or 94% thyroid cancer clinical sensitivity. In embodiments of the invention the clinical sensitivity of a method of the invention can be greater than about 80 to 90%, more particularly greater than about 80 to 85%, most particularly greater than about 83%, 84%, or 85%. Clinical sensitivity and specificity may be determined using methods known to persons skilled in the art.

In accordance with methods of the invention, a Thyroid Cancer Marker in a sample can be assessed by detecting the presence in the sample of (a) a polypeptide or polypeptide fragment corresponding to the marker; (b) a transcribed nucleic acid or fragment thereof having at least a portion with which the marker is substantially identical; and/or (c) a transcribed nucleic acid or fragment thereof, wherein the nucleic acid hybridizes with the marker.

One aspect of the invention provides a method for detecting or characterizing a thyroid cancer in a patient comprising determining the status of Thyroid Cancer Markers in a sample obtained from the patient, wherein an abnormal status in the sample indicates the presence of the condition. Another aspect of the invention provides a method of screening for thyroid cancer in a patient comprising identifying a patient at risk of having thyroid cancer or in need of screening and determining the status of Thyroid Cancer Markers in a sample obtained from the patient, wherein an abnormal status of the markers indicates the presence of thyroid cancer. In some embodiments, the patient is at risk of developing a specific type of thyroid cancer and the abnormal status indicates the presence of the specific type of thyroid cancer.

Another aspect provides a diagnostic method comprising identifying a patient who is a candidate for treatment for thyroid cancer and determining the status of Thyroid Cancer Markers in a sample obtained from the patient, wherein an abnormal status of the markers in the sample indicates that treatment is desirable or necessary.

In aspects of the invention, the abnormal status can be an elevated status, low status or negative status. In an embodiment of the invention for detecting or diagnosing thyroid cancer or a type of thyroid cancer the abnormal status is an elevated status.

In an aspect of the invention, a method is provided for detecting Thyroid Cancer Markers associated with thyroid cancer in a patient comprising or consisting essentially of: (a) obtaining a sample from a patient; b) detecting or identifying in the sample one or more Thyroid Cancer Markers, in particular a Thyroid Cancer Marker set out in Table 1; and (c) comparing the detected amount with an amount detected for a standard.

In an aspect, the invention provides a method for diagnosing or screening for thyroid cancer in a subject, the method comprising: (a) contacting a sample from a subject with reagents capable of measuring levels of target Thyroid Cancer Markers, in particular Thyroid Cancer Markers set out in Table 1; and (b) providing a diagnosis of thyroid cancer in said subject based on a significant difference in the level of the Thyroid Cancer Markers in the sample from the subject over a control level obtained from similar samples taken from subjects who do not have thyroid cancer or from the subject at a different time.

In an embodiment of the invention, a method is provided for detecting one or more of the Thyroid Cancer Markers in a patient comprising or consisting essentially of: (a) obtaining a sample from a patient; (b) detecting or identifying in the sample one or more of the Thyroid Cancer Markers, in particular Thyroid Cancer Markers set out in Table 1; and (c) comparing the detected amounts with amounts detected for a standard.

In embodiments of the invention, the Thyroid Cancer Markers are chosen from tyrosine-protein kinase receptor UFO (AXL) [SEQ ID NO. 1], activated leukocyte cell adhesion molecule (ALCAM)/CD166 [SEQ ID NO. 2], and prothymosin alpha (PTMA) [SEQ ID NO. 3], and optionally galectin-3 [SEQ ID NO. 4]. In embodiments of the invention, the Thyroid Cancer Markers comprise or are selected from the group consisting of or consisting essentially of tyrosine-protein kinase receptor UFO (AXL) and activated leukocyte cell adhesion molecule (ALCAM)/CD 166.

In embodiments of the invention, the Thyroid Cancer Markers comprise or are selected from the group consisting of or consisting essentially of tyrosine-protein kinase receptor UFO (AXL), activated leukocyte cell adhesion molecule (ALCAM)/CD166, and prothymosin alpha (PTMA).

In embodiments of the invention, the Thyroid Cancer Marker is biotinidase [SEQ ID NO. 5]. In embodiments of the invention, the Thyroid Cancer Markers comprises clusterin [SEQ ID NO. 6], activated leukocyte cell adhesion molecule (ALCAM)/CD166, amyloid precursor protein like protein 2 (APLP2) [SEQ ID NO.7], tyrosine-protein kinase receptor UFO (AXL), nucleolin [SEQ ID NO. 8], PTMA, amyloid precursor protein (APP) [SEQ ID NO. 9], 14-3-3 zeta [SEQ ID NO. 10], SET [SEQ ID NO.11], PKM2 [SEQ ID NO. 12] or hnRNPK [SEQ ID NO. 13].

In embodiments of the invention, the Thyroid Cancer Markers comprise or are selected from the group consisting of or consisting essentially of tyrosine-protein kinase receptor UFO (AXL), activated leukocyte cell adhesion molecule (ALCAM)/CD166, prothymosin-alpha (PTMA), nucleolin, biotinidase, amyloid precursor protein (APP), APLP2 and calsyntenin-1 [SEQ ID NO. 14].

In embodiments of the invention, the Thyroid Cancer Markers measured additionally comprise or are selected from the group consisting of or consisting essentially of essentially of clusterin, Dickkopf-related protein 3 (DKK-3) [SEQ ID NO. 15], nidogen-1 [SEQ ID NO. 16], gelsolin [SEQ ID NO. 17], and nucleobindin [SEQ ID NO. 18].

In embodiments of the invention, the Thyroid Cancer Markers measured additionally comprise or are selected from the group consisting essentially of CYR61 [SEQ ID NO. 19], E-cadherin [SEQ ID NO. 20] and prothymosin-alpha.

In embodiments of the invention, the Thyroid Cancer Markers measured additionally comprise or are selected from the group consisting essentially of α-Enolase [SEQ ID NO. 21], and dystroglycan 1 [SEQ ID NO. 22].

In embodiments of the invention, the Thyroid Cancer Markers measured additionally comprised or selected from the group consisting essentially of clusterin, Dickkopf-related protein 3 (DKK-3), nidogen-1, gelsolin, nucleobindin, melanoma-associated antigen [SEQ ID NO. 23], osteopontin [SEQ ID NO. 24] and plasminogen activator urokinase [SEQ ID NO. 25].

In embodiments of the invention, the Thyroid Cancer Markers measured additionally comprise or are selected from the group consisting essentially of clusterin, Dickkopf-related protein 3 (DKK-3), nidogen-1, gelsolin, nucleobindin, melanoma-associated antigen, osteopontin, plasminogen activator urokinase, nucleotin, CYR61, E-cadherin and prothymosin-alpha.

In embodiments of this aspect of the invention, the Thyroid Cancer Markers measured comprise two, three, four, five, six, seven, eight, nine, ten or more markers set out in Table 1.

The invention further provides a non-invasive non-surgical method for detection or diagnosis of thyroid cancer in a subject comprising: obtaining a sample (e.g., fluid sample) from the subject; subjecting the sample to a procedure to detect Thyroid Cancer Marker(s); detecting or diagnosing thyroid cancer by comparing the levels of Thyroid Cancer Marker(s) to the levels of Thyroid Cancer Marker(s) obtained from a control subject with no thyroid cancer or a lower grade of thyroid cancer. In embodiments of this method of the invention, the Thyroid Cancer Marker(s) are one or more of the markers set out in Table 1.

The invention contemplates a method for determining the aggressiveness or stage of thyroid cancer comprising producing a profile of levels of Thyroid Cancer Markers, and other markers associated with thyroid cancer, in cells from a patient, and comparing the profile with a reference to identify a profile for the test cells indicative of aggressiveness or stage of disease. In an aspect, the markers are Polypeptide Thyroid Cancer Markers and the profile is generated using a mass spectrometer.

In particular aspects, methods of the invention are used to diagnose the stage of thyroid cancer in a subject or characterizing thyroid cancer in a subject. In an embodiment, the method comprises comparing: (a) levels of one or more Thyroid Cancer Markers set out in Table 1, in particular the follicular thyroid cancer markers, papillary thyroid cancer markers or aggressive/metastatic thyroid cancer markers set out in Table 1, from a sample from the patient; and (b) levels of the Thyroid Cancer Markers in control samples of the same type obtained from patients without thyroid cancer or control patients with a different stage of thyroid cancer (e.g., low grade thyroid cancer) or from another sample from the subject, wherein altered levels of Thyroid Cancer Markers, relative to the corresponding levels in the control samples is an indication that the patient is afflicted with a more aggressive or metastatic thyroid cancer.

In embodiments, follicular thyroid cancer is diagnosed and the Thyroid Cancer Markers are one or more of the Thyroid Cancer Markers for follicular thyroid cancer set out in Table 1.

In aspects of the invention, a method is provided for diagnosing follicular thyroid cancer in a patient comprising or consisting essentially of: (a) detecting or identifying in the sample one, two or three of the Thyroid Cancer Markers for follicular thyroid cancer set out in Table 1, and optionally one or more additional Thyroid Cancer Marker set out in Table 1 or 2; and (b) comparing the detected amount with an amount detected for a standard, wherein a significant difference in the Thyroid Cancer Markers is indicative of follicular thyroid cancer. In embodiments of the invention relating to follicular thyroid cancer, the Thyroid Cancer Markers comprise or are selected from the group consisting essentially of calmodulin [SEQ ID NO. 26], CD44 antigen [SEQ ID NO. 27], fibronectin [SEQ ID NO. 28], ubiquitin A-52 residue ribosomal protein fusion product [SEQ ID NO. 29], and basement membrane specific heparin sulfate core protein [SEQ ID NO. 30].

In embodiments, papillary thyroid cancer is diagnosed and the Thyroid Cancer Markers are one or more of the Thyroid Cancer Markers for papillary thyroid cancer set out in Table 1. In a particular embodiment of the invention, a method is provided for diagnosing or detecting papillary thyroid cancer in a patient comprising or consisting essentially of: (a) detecting or identifying in the sample one or more Thyroid Cancer Markers for papillary thyroid cancer set out in Table 1, and optionally one or more additional Thyroid Cancer Marker set out in Table 1 and 2; and (b) comparing the detected amount with an amount detected for a standard, wherein significant difference in the amount of the Thyroid Cancer Markers is indicative of papillary thyroid cancer. In embodiments of the invention relating to papillary thyroid cancer, the Thyroid Cancer Markers comprise versican [SEQ ID NO. 31], nucleolin and/or prothymosin-alpha. In embodiments of the invention relating to papillary thyroid cancer, the Thyroid Cancer Markers comprise versican, nucleolin and/or prothymosin-alpha, and optionally CYR61 and/or E-cadherin.

In embodiments of the invention relating to papillary thyroid cancer, the Thyroid Cancer Markers comprise one or more of, or are selected from the group consisting of or consisting essentially of gamma-glutamyl hydrolase [SEQ ID NO. 32], lysyl oxidase-like 2 [SEQ ID NO. 33], biotinidase, and nidogen-1. The Thyroid Cancer Markers for detecting papillary thyroid cancer may additionally comprise cysteine-rich angiogenic inducer, 61 (CYR61) and/or E-cadherin. In embodiments of the invention relating to papillary thyroid cancer, the Thyroid Cancer Markers comprise two, three, four, five, six, seven, eight, nine, ten or all the for papillary thyroid cancer markers set out in Table 1.

In embodiments, aggressive thyroid cancer, in particular ATC, is diagnosed and the Thyroid Cancer Markers are one or more of the Thyroid Cancer Markers for aggressive/metastatic thyroid cancer set out in Table 1. In a particular aspect of the invention, a method is provided for detecting Thyroid Cancer Markers associated with aggressive or metastatic thyroid cancer, in a patient comprising or consisting essentially of: (a) obtaining a sample from a patient; (b) detecting in the sample one or more Thyroid Cancer Markers for aggressive/metastatic thyroid cancer set out in Table 1 and optionally one or more additional Thyroid Cancer Marker set out in Tables 1 and 2; and (c) comparing the detected amount with an amount detected for a standard or cut-off value. In embodiments of the invention relating to aggressive or metastatic thyroid cancer, the Thyroid Cancer Markers comprise two, three, four or all the aggressive/metastatic thyroid cancer markers set out in Table 1. In embodiments of the invention relating to aggressive or metastatic thyroid cancer, the Thyroid Cancer Markers comprise prothymosin-alpha. In embodiments of the invention relating to aggressive or metastatic thyroid cancer, the Thyroid Cancer Markers comprise one or more or all of, or are selected from the group consisting essentially of activated leukocyte cell adhesion molecule (ALCAM)/CD166, tyrosine-protein kinase receptor UFO (AXL), amyloid precursor protein like protein 2 (APLP2), cadherin-2, prothymosin-alpha, clusterin, syndecan-4 [SEQ ID NO. 34], E-cadherin, gelsolin, hnRNP A2/B1 [SEQ ID NO. 35], nucleolin, α-MCFD2 [SEQ ID NO. 36], α-NPC2 [SEQ ID NO. 37] and SET protein.

The invention provides a method of assessing whether a patient is afflicted with thyroid cancer, the method comprising comparing: (a) levels of one or more Thyroid Cancer Markers set out in Table 1 from the patient; and (b) standard levels of Thyroid Cancer Markers in samples of the same type obtained from control patients not afflicted with thyroid cancer or with a lower grade of thyroid cancer, wherein altered levels of Thyroid Cancer Markers relative to the corresponding standard levels of Thyroid Cancer Markers is an indication that the patient is afflicted with thyroid cancer. In an embodiment of a method of the invention for assessing whether a patient is afflicted with follicular thyroid cancer (FTC), levels of one or more Thyroid Cancer Markers for follicular thyroid cancer set out in Table 1, in particular basement membrane specific heparin sulfate core protein, in a sample from the patient are compared to a standard. In an embodiment of a method of the invention for assessing whether a patient is afflicted with follicular thyroid cancer (FTC), levels of one or more Thyroid Cancer Markers for follicular thyroid cancer set out in Table 1 and optionally one or more additional Thyroid Cancer Marker set out in Tables 1 and 2, in particular calmodulin, CD44 antigen, fibronectin, ubiquitin A-52 residue ribosomal protein fusion product, and basement membrane specific heparin sulfate core protein, in a sample from the patient are compared to a standard.

In an embodiment of a method of the invention for assessing whether a patient is afflicted with follicular thyroid cancer higher levels of one or more Thyroid Cancer Markers for follicular thyroid cancer set out in Table 1 and optionally one or more additional Thyroid Cancer Marker set out in Tables 1 and 2, in particular calmodulin, CD44 antigen, fibronectin, ubiquitin A-52 residue ribosomal protein fusion product, or basement membrane specific heparin sulfate core protein, in a sample relative to a standard or corresponding normal levels, is an indication that the patient is afflicted with follicular thyroid cancer.

In an embodiment of a method of the invention for assessing whether a patient is afflicted with papillary thyroid cancers (PTC), levels of one or more Thyroid Cancer Markers for papillary thyroid cancer set out in Table 1 in a sample from the patient are compared to a standard. In an embodiment of a method of the invention for assessing whether a patient is afflicted with papillary thyroid cancers (PTC), levels of nucleolin and/or prothymosin-alpha, and optionally CYR61 and/or E-cadherin, in a sample from the patient are compared to a standard.

In an aspect of a method of the invention for assessing whether a patient is afflicted with papillary thyroid cancer higher levels of one or more Thyroid Cancer Markers for papillary thyroid cancer set out in Table 1 in a sample relative to a standard or corresponding normal levels, is an indication that the patient is afflicted with follicular thyroid cancer. In an aspect of a method of the invention for assessing whether a patient is afflicted with papillary thyroid cancer higher levels of one or both of nucleolin and/or prothymosin-alpha, and optionally CYR61 and/or E-cadherin, in a sample relative to a standard or corresponding normal levels, is an indication that the patient is afflicted with follicular thyroid cancer.

In an embodiment of a method of the invention for assessing whether a patient is afflicted with aggressive or metastatic thyroid cancer, levels of one or more Thyroid Cancer Markers for aggressive or metastatic thyroid cancer set out in Table 1 in a sample from the patient are compared to a standard. In an aspect of a method of the invention for assessing whether a patient is afflicted with aggressive or metastatic thyroid cancer higher levels of one or more Thyroid Cancer Markers aggressive or metastatic thyroid cancer set out in Table 1 in a sample relative to a standard or corresponding normal levels or levels from a patient with a lower grade of thyroid cancer, is an indication that the patient is afflicted with aggressive or metastatic thyroid cancer.

In an embodiment of a method of the invention for assessing whether a patient is afflicted with anaplastic thyroid cancer, levels of one or more Thyroid Cancer Markers for aggressive or metastatic thyroid cancer set out in Table 1 and particularly marked * * * in a sample from the patient are compared to a standard, and significantly different levels of the Thyroid Cancer Markers compared to a standard are indicative of anaplastic thyroid cancer.

In an aspect of a method of the invention for assessing whether a patient is afflicted with aggressive or metastatic thyroid cancer or anaplastic thyroid cancer levels of prothymosin-alpha in a sample from the patient are compared to a standard.

In aspects of the invention, aggressive thyroid cancer, in particular ATC, is detected, diagnosed or characterized by determination of increased levels of one or more Thyroid Cancer Marker(s) aggressive or metastatic thyroid cancer set out in Table 1 and Marked * * * when compared to such levels obtained from a control.

In an aspect, the invention provides a method for monitoring the progression of thyroid cancer in a patient the method comprising: (a) detecting one or more Thyroid Cancer Marker(s) set out in Table 1 and optionally Table 2 in a patient sample (e.g. biopsy sample) at a first time point; (b) repeating step (a) at a subsequent point in time; and (c) comparing the levels detected in (a) and (b), and thereby monitoring the progression of thyroid cancer in the patient.

The invention provides a method for classifying a patient having thyroid cancer, the method comprising measuring one or more Thyroid Cancer Marker(s) set out in Table 1 and optionally Table 2 in a fluid sample, in particular serum sample, from the patient and correlating the values measured to values measured for the Thyroid Cancer Markers from thyroid cancer patients stratified in classification groups. The method can be used to predict patient survival, wherein the Thyroid Cancer Marker(s) are predictive of survival and wherein the classification groups comprise groups of known overall survival. In aspects of this method of the invention, the Thyroid Cancer Marker(s) are selected from the follicular thyroid cancer markers, papillary thyroid cancer markers or aggressive/metastatic thyroid cancer markers in Table 1. In various embodiments the values measured can be normalized to provide more accurate quantification and to correct for experimental variations.

In aspects of the invention, Polynucleotide Thyroid Cancer Markers are detected and levels of Polynucleotide Thyroid Cancer Markers in a sample from a patient are compared with Polynucleotide Thyroid Cancer Marker levels from samples of patients without thyroid cancer, with a lower grade of thyroid cancer, or from levels from samples of the same patient. A method of the invention may employ one or more polynucleotides, oligonucleotides, or nucleic acids capable of hybridizing to Polynucleotide Thyroid Cancer Markers. The present invention relates to a method for diagnosing and characterizing thyroid cancer, more particularly the stage of thyroid cancer, in a sample from a subject comprising isolating nucleic acids, preferably mRNA, from the sample, and detecting Polynucleotide Thyroid Cancer Markers in the sample.

The invention also provides methods for determining the presence or absence of thyroid cancer or the aggressiveness or metastatic potential of a thyroid cancer in a subject in the subject comprising detecting in the sample a level of nucleic acids that hybridize to one or more Polynucleotide Thyroid Cancer Marker(s) encoding polypeptides set out in Table 1, and optionally Table 2, and comparing the level(s) with a predetermined standard or cut-off value, and therefrom determining the presence or absence of thyroid cancer or the aggressiveness or metastatic potential of a thyroid cancer in the subject in the subject. In an embodiment a method is provided for determining the aggressiveness or metastatic potential of thyroid cancer in a subject comprising (a) contacting a sample taken from the subject with oligonucleotides that hybridize to one or more polynucleotides encoding the Thyroid Cancer Markers for follicular thyroid cancer markers, papillary thyroid cancer markers and/or aggressive/metastatic thyroid cancer markers set out in Table 1; and (b) detecting in the sample a level of nucleic acids that hybridize to the oligonucleotides relative to a predetermined standard or cut-off value, and therefrom determining the aggressiveness or metastatic potential of the cancer in the subject.

In an aspect, the invention provides a method of assessing the aggressiveness or metastatic potential of a thyroid cancer in a patient, the method comprising comparing: (a) levels of one or more Polynucleotide Thyroid Cancer Marker(s) set out in Table 1, in particular follicular thyroid cancer markers, papillary thyroid cancer markers or aggressive/metastatic thyroid cancer markers set out in Table 1, in a sample from the patient; and (b) control levels of the Polynucleotide Thyroid Cancer Marker(s) in samples of the same type obtained from control patients not afflicted with thyroid cancer or a lower grade of thyroid cancer, wherein altered levels of Polynucleotide Thyroid Cancer Marker(s) relative to the corresponding control levels of the Polynucleotide Thyroid Cancer Marker(s) is an indication of the aggressiveness or metastatic potential of the thyroid cancer.

In a particular method of the invention for assessing whether a patient is afflicted with an aggressive or metastatic thyroid cancer higher levels of prothymosin-alpha or nucleolin, in a sample relative to the corresponding control levels is an indication that the patient is afflicted with an aggressive or metastatic thyroid cancer.

Within certain embodiments, the amount of nucleic acid that is mRNA is detected via amplification reactions such as polymerase chain reaction (PCR) using, for example, at least one oligonucleotide primer that hybridizes to a Polynucleotide Thyroid Cancer Marker(s) or a complement of such polynucleotide. Within other embodiments, the amount of mRNA is detected using a hybridization technique, employing an oligonucleotide probe that hybridizes to a Polynucleotide Thyroid Cancer Marker(s), or a complement thereof.

When using mRNA detection, the method may be carried out by combining isolated mRNA with reagents to convert to cDNA according to standard methods; treating the converted cDNA with amplification reaction reagents along with an appropriate mixture of primers to produce amplification products; and analyzing the amplification products to detect the presence of Polynucleotide Thyroid Cancer Marker(s) in the sample. For mRNA the analyzing step may be accomplished using RT-PCR analysis to detect the presence of Polynucleotide Thyroid Cancer Marker(s). The analysis step may be accomplished by quantitatively detecting the presence of Polynucleotide Thyroid Cancer Marker(s) in the amplification product, and comparing the quantity of Polynucleotide Thyroid Cancer Marker(s), detected against a panel of expected values for known presence or absence in normal and malignant samples derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a Polynucleotide Thyroid Cancer Marker(s) to produce amplification products; (d) analyzing the amplification products to detect an amount of mRNA Polynucleotide Thyroid Cancer Marker(s); and (e) comparing the amount of mRNA to an amount detected against a panel of expected values for normal and malignant samples (e.g., samples from patients with a different stage of thyroid cancer) derived using similar nucleic acid primers.

In particular embodiments of the invention, the methods described herein utilize the Polynucleotide Thyroid Cancer Markers placed on a microarray so that the expression status of each of the markers is assessed simultaneously. In a particular aspect, the invention provides a microarray comprising a defined set of genes whose expression is significantly altered by a thyroid cancer or procedure. The invention further relates to the use of the microarray as a prognostic tool to predict thyroid cancer or status of a thyroid cancer. In an embodiment, the invention provides for oligonucleotide arrays comprising marker sets described herein. The microarrays provided by the present invention may comprise probes to markers able to distinguish thyroid cancer. In particular, the invention provides oligonucleotide arrays comprising probes to a subset or subsets of at least 5 or 10 gene markers up to a full set of markers which distinguish thyroid cancer.

Protein based methods can also be used for determining the presence or absence of thyroid cancer or the aggressiveness or metastatic potential of a thyroid cancer in a sample from a subject. Thyroid Cancer Markers may be detected using a binding agent for Thyroid Cancer Markers, preferably antibodies specifically reactive with Thyroid Cancer Markers, or parts thereof.

The invention provides a method of assessing whether a patient is afflicted with thyroid cancer which comprises comparing: (a) levels of one or more Polypeptide Thyroid Cancer Markers set out in Table 1, and optionally Table 2 in a sample from the patient; and (b) control levels of the Polypeptide Thyroid Cancer Markers in a non-cancer sample or sample from a patient with a lower grade of thyroid cancer or from a sample from the patient taken at another time, wherein significantly different levels of Polypeptide Thyroid Cancer Markers in the sample from the patient compared with the control levels is an indication that the patient is afflicted with thyroid cancer.

In another aspect the invention provides methods for determining the presence or absence of thyroid cancer or the aggressiveness or metastatic potential of a thyroid cancer or classifying thyroid cancer in a patient comprising the steps of (a) contacting a biological sample obtained from a patient with a binding agent that specifically binds to one or more Polypeptide Thyroid Cancer Marker(s) set out in Table 1 and optionally Table 2; and (b) detecting in the sample an amount of the Polypeptide Thyroid Cancer Marker(s) that binds to the binding agent(s), relative to a predetermined standard or cut-off value, and therefrom determining the presence or absence of thyroid cancer, the aggressiveness or metastatic potential of thyroid cancer or the stage of thyroid cancer in the patient.

In an embodiment, the invention relates to a method for detecting, diagnosing, staging and monitoring thyroid cancer in a subject by quantitating one or more Polypeptide Thyroid Cancer Marker(s) in a biological sample from the subject comprising (a) reacting the biological sample with an antibody specific for one or more Polypeptide Thyroid Cancer Marker(s) set out in Table 1, and optionally Table 2, which is directly or indirectly labeled with a detectable substance; and (b) detecting the detectable substance.

In another embodiment the invention provides a method of using antibodies to detect expression of Polypeptide Thyroid Cancer Marker(s) in a sample, the method comprising: (a) combining antibodies specific for Polypeptide Thyroid Cancer Marker(s) with a sample under conditions which allow the formation of antibody:protein complexes; and (b) detecting complex formation, wherein complex formation indicates expression of Polypeptide Thyroid Cancer Marker(s) in the sample. Expression may be compared with standards and is diagnostic of thyroid cancer, stage of thyroid cancer, or the aggressiveness or metastatic potential of the thyroid cancer.

Polypeptide Thyroid Cancer Markers can be determined by constructing an antibody microarray in which binding sites comprise immobilized, preferably monoclonal, antibodies specific to a substantial fraction of marker-derived thyroid cancer proteins of interest.

In an aspect, the invention provides a method for monitoring the progression of thyroid cancer in a patient, the method comprising: (a) detecting one or more Polypeptide Thyroid Cancer Marker(s) set out in Table 1, and optionally Table 2, in a patient sample at a first time point; and (b) repeating step (a) at a subsequent point in time; and (c) comparing the levels detected in (a) and (b), and thereby monitoring the progression of thyroid cancer in the patient.

The invention further relates to a method of assessing the efficacy of a therapy for thyroid cancer in a patient. This method comprises comparing: (a) levels of one or more Thyroid Cancer Markers set out in Table 1, and optionally Table 2, in a first sample obtained from the patient prior to providing at least a portion of the therapy to the patient; and (b) levels of the Thyroid Cancer Markers in a second sample obtained from the patient following therapy. Significantly different levels of Thyroid Cancer Markers in the second sample, relative to the first sample, can be an indication that the therapy is efficacious for inhibiting thyroid cancer, In an embodiment, the method is used to assess the efficacy of a therapy for inhibiting thyroid cancer and significantly different levels of one or more Thyroid Cancer Markers for follicular thyroid cancer, papillary thyroid cancer and/or aggressive/metastatic thyroid cancer in Table 1, in the second sample relative to the first sample, is an indication that the therapy is efficacious for inhibiting the cancer or metastasis. The therapy may be any therapy for treating thyroid cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden, aggressiveness or metastatic potential of the tumor.

The invention provides marker sets for diagnosing or characterizing thyroid cancer and uses thereof. A marker set may comprise a plurality of polypeptides and/or polynucleotides encoding such polypeptides comprising or consisting of at least 2, 3, 4, 5, 6, 7, 8, 9, 10, 11, 12, 13, 14, or 15 of the markers of Table 1. In specific aspects, the markers consist of at least 2, 3, 4, 5, 6, 7, 8, 9, or 10 polypeptides of Table 1. In an aspect the protein marker sets comprise or consist of protein clusters, or proteins in pathways comprising markers of Table 1. In embodiments of the invention, a marker is provided which is selected from the group consisting of the polypeptides set forth in Table 1 which polypeptides are up-regulated biomarkers in thyroid cancer, in particular follicular thyroid cancer, papillary cancer or metastatic/aggressive thyroid cancer.

The invention also provides a diagnostic composition comprising Thyroid Cancer Markers or agents that interact with Thyroid Cancer Markers. In particular, the invention provides a diagnostic composition comprising Polypeptide Thyroid Cancer Markers, or agents that bind to such markers, or hybridize to or amplify Polynucleotide Thyroid Cancer Markers. In an embodiment, the composition comprises a probe that specifically hybridizes to a Polynucleotide Thyroid Cancer Marker or a fragment thereof, and a probe that specifically hybridizes to a Polynucleotide Thyroid Cancer Marker or a fragment thereof. In another embodiment a composition is provided comprising a specific primer(s) pair capable of amplifying a Polynucleotide Thyroid Cancer Marker using polymerase chain reaction methodologies. In a still further embodiment, the composition comprises a binding agent(s) (e.g. antibody) that binds to a Polypeptide Thyroid Cancer Marker or a fragment thereof. Probes, primers, and binding agents can be labeled with a detectable substance. In an embodiment, a diagnostic composition of the invention comprises one or more antibodies specific for a Thyroid Cancer Marker in Table 1. In an embodiment, a diagnostic composition of the invention comprises one or more primers that amplify polynucleotides encoding a Thyroid Cancer Marker in Table 1.

In another aspect, the invention relates to use of an agent that interacts with a Thyroid Cancer Marker in the manufacture of a composition for diagnosing thyroid cancer, in particular the aggressiveness or metastatic potential of a thyroid cancer.

The methods of the invention may also comprise detecting additional markers associated with thyroid cancer, for example the markers listed in Table 2, more particularly galectin-3, thyroglobulin, and E-cadherin. Further, the amount of Thyroid Cancer Markers may be mathematically combined with other markers of thyroid cancer. In an embodiment the invention provides a method for detecting or diagnosing thyroid cancer in a subject comprising: (a) determining the amount of one or more Thyroid Cancer Markers in Table 1 in a sample from the subject; (b) determining the amount of other markers associated with thyroid cancer in particular markers comprising or selected from the markers listed in Table 2 or from the group consisting of or consisting essentially of galectin-3, thyroglobulin and E-cadherin, in the sample; (c) mathematically combining the results of step (a) and step (b) to provide a mathematical combination; and (d) comparing or correlating the mathematical combination to the presence of thyroid cancer, stage of thyroid cancer or aggressiveness or metastatic potential of thyroid cancer.

In a particular embodiment the invention provides a method for diagnosing the aggressiveness of thyroid cancer in a subject comprising: (a) determining the amount of one or more Thyroid Cancer Marker(s) for aggressive or metastatic thyroid cancer set out in Table 1 from the subject; (b) determining the amount of Thyroid Cancer Marker(s) in the sample; (c) determining the amount of one or more of E-cadherin, CYR61, melanoma-associated antigen, osteopontin, and plasminogen activator urokinase in the sample; (d) mathematically combining the results of step (a) and step (b), and optionally step (c) to provide a mathematical combination; and (e) comparing or correlating the mathematical combination to the aggressiveness of the thyroid cancer. The combination is preferably compared to a mathematical combination for a predetermined standard. In particular aspects, the invention provides a method for detecting, characterizing or diagnosing thyroid cancer by determining the combination of Thyroid Cancer Markers and one or more of the markers listed in Table 2, or one or both of galectin-3 and thyroglobulin in a sample from a subject.

The invention also includes kits for carrying out methods of the invention. In an aspect the invention provides a kit for detecting, diagnosing, screening for, monitoring, predicting or characterizing thyroid cancer comprising Thyroid Cancer Markers. In a particular aspect, the invention provides a test kit for diagnosing screening for, monitoring, predicting or characterizing thyroid cancer in a subject which comprises an agent that interacts with a Thyroid Cancer Marker(s). In an embodiment, the kit is for assessing whether a patient is afflicted with follicular thyroid cancer and it comprises reagents for identifying and/or assessing levels of the Thyroid Cancer Markers for follicular thyroid cancer in Table 1. In an embodiment, the kit is for assessing whether a patient is afflicted with papillary thyroid cancer and it comprises reagents for identifying and/or assessing levels of the Thyroid Cancer Markers for papillary thyroid cancer in Table 1. In an embodiment, the kit is for assessing whether a patient is afflicted with aggressive or metastatic thyroid cancer and it comprises reagents for identifying and/or assessing levels of the Thyroid Cancer Markers for aggressive or metastatic thyroid cancer in Table 1.

The invention contemplates an in vivo method comprising administering to a mammal one or more agent that carries a label for imaging and binds to a Thyroid Cancer Marker, and then imaging the mammal. According to a preferred aspect of the invention, an in vivo method for imaging thyroid cancer is provided comprising: (a) injecting a patient with an agent that binds to a Thyroid Cancer Marker(s), the agent carrying a label for imaging the thyroid cancer; (b) allowing the agent to incubate in vivo and bind to the Thyroid Cancer Marker(s); and (c) detecting the presence of the label localized to the thyroid cancer. In an embodiment of the invention the agent is an antibody which recognizes the Thyroid Cancer Marker(s). In another embodiment of the invention the agent is a chemical entity which recognizes the Thyroid Cancer Marker(s). The agent carries a label to image the Thyroid Cancer Marker(s). Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The invention also contemplates the localization or imaging methods described herein using multiple markers for thyroid cancer.

In an aspect, the invention provides antagonists (e.g. antibodies) specific for a Thyroid Cancer Marker set out in Table 1 that can be used therapeutically to destroy or inhibit the growth of thyroid cancer cells.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows the verification of secreted proteins in thyroid cancer patients' sera by immunoblotting in tissue culture media and sera of thyroid cancer patients. Protein samples were prepared from cultured cells (50-75 μg), their respective serum-free media (SFM; 5 μg), and the sera (50 μg) of five thyroid cancer patients (2 non-aggressive PTC, FTC, aggressive PTC (metastatic thyroid cancer) and ATC and one normal sample (‘normal’, S-2145, Sigma). As protein loading controls, lysates were examined for actin expression, while SFM and patients' sera were stained with Ponceau S (Sigma). Western blot analysis confirmed the detection of five proteins, namely clusterin, CYR 61, α-enolase, nucleolin and prothymosin-alpha in the thyroid carcinoma patients' sera and in the whole-cell lysate and conditioned media (CM) of the thyroid carcinoma cell lines as shown in panel A. The panel B shows increase in α-MCFD2 [SEQ ID NO. 36], α-NPC2 [SEQ ID NO. 37], E cadherin and calsyntenin1, in normal and thyroid cancer patients' sera. The panels C and D show decrease in α-Enolase probed with rabbit polyclonal antibody (H300; Santa Cruz Biotech) and a mouse monoclonal antibody (ab54979; Abeam). Levels of the 48-kDa α-Enolase protein expressed relative to the level of this protein in benign thyroid nodule taken as 1.0 were observed to decrease in ATC and metastatic PTC suggesting a reduction that correlated with disease aggressiveness. FIG. 1 panels E, F and G show immunohistochemical analysis of α-Enolase expression in non-aggressive PTC (E), metastatic PTC (F), and normal thyroid tissue (G) (Original magnification X400). Reduced cytoplasmic and nuclear staining was observed in PTC and metastatic PTC as compared to the normal thyroid tissues; reduction was more pronounced in the aggressive PTC tissue.

FIG. 2, panels B and C show increased nuclear and cytoplasmic expression of prothymosin alpha (PTMA) by immunohistochemical analysis in human thyroid carcinoma tissues—(B) PTC and (C) ATC as compared to the normal tissue (A) (Original magnification X400). Panel D shows increased nuclear nucleolin expression in normal thyroid tissue, while the PTC (E) and ATC (F) showed reduced nuclear and increased cytoplasmic expression of nucleolin (Original magnification X400). Panels G and H depict scatter plot analysis of immunohistochemical scoring of PTMA and nucleolin in different subtypes of thyroid carcinomas. Tissues were scored based on percentage positivity and immunostaining intensity. Sections were scored as positive if epithelial cells showed immunoreactivity in the plasma membrane, cytoplasm, and/or nucleus when observed by evaluators blinded to the clinical history and outcome. The percentage positive scores were assigned according to the following scale: 0, <10% cells; 1, 10-30% cells; 2, 30-50% cells; 3, 50-70% cells; and 4, >70%. The intensity of the staining was also scored semi-quantitatively as follows: 0, no staining; 1, mild; 2, moderate; and 3, intense. The total score (0-7) was obtained by adding the percentage positivity scores and intensity scores for each section. (G) ATC displayed elevated nuclear expression of PTMA compared to normal thyroid adjacent to benign thyroid disease, insular and papillary thyroid carcinomas. ATC cases displayed strikingly elevated cytoplasmic expression of PTMA compared to PTC and insular cells. PTC staining of cytoplasmic PTMA was low. Insular (poorly differentiated) thyroid carcinomas also demonstrated an increased expression of cytoplasmic PTMA compared to PTC and normal thyroid adjacent to benign thyroid disease. (H) Nuclear expression of nucleolin was seen in all thyroid carcinoma subtypes and normal thyroid adjacent to benign thyroid disease examined. Faint cytoplasmic expression was also observed in ATC cases only. These findings suggest increase in nuclear and cytoplasmic expression of PTMA with disease aggressiveness, while increased cytoplasmic nucleolin was observed in ATC.

FIG. 3, panel A, B and C show detection and verification of serum biotinidase in patients with thyroid cancer. Serum proteins from patients with benign (n=8), non-aggressive PTC (n=6) and metastatic PTC (n=6) were immunodepleted, resolved on SDS-PAGE and probed with α-biotinidase rabbit polyclonal antibody K-17 (Santa Cruz Biotech; 1:1000 dilution) or mouse monoclonal (ab54886; Abcam) antibody. The 75 kDa biotinidase protein band levels were quantified using ImageQuant software (GE Biosciences) and are expressed as a proportion of the highest benign band intensity per blot. Biotinidase levels decreased from benign levels for nearly all of the PTC cases that correlated with disease aggressiveness. Panels D-G show immunohistochemical analysis of biotinidase expression in thyroid tissues. Stronger cytoplasmic staining was observed in the non-aggressive (E) and metastatic PTC (F), whereas nuclear levels of biotinidase decreased with increasing thyroid cancer aggressiveness. Original magnification ×400. Scatter plot analysis of biotinidase cytoplasmic and nuclear expression is shown in panels H and I, benign (n=3), non-aggressive (n=21) and aggressive (n=6) PTC cases with respective mean and standard error bars indicated. Stronger cytoplasmic staining above the benign levels was observed for all non-aggressive and aggressive thyroid cancer cases. In the nucleus, biotinidase expression was found to decrease with increased tumor aggressiveness.

FIG. 4, panels A and B show verification of serum clusterin levels in thyroid cancer patients. Serum proteins from patients with benign (n=8), non-aggressive (n=6) and aggressive (n=6) PTC were immunodepleted treatment prior to electrophoresis. To detect clusterin protein, blots were prepared and probed with α-clusterin mouse monoclonal antibody (78E; Santa Cruz Biotech) at a 1:2000 dilution. The expected 40-kDa protein was detected by 78E and found to decrease in abundance from benign levels in both non-aggressive and metastatic PTC cases. This reduction was also more pronounced in patients with more aggressive disease. Panel B shows verification of serum clusterin with mouse and rabbit α-clusterin antibodies. Immunoblots containing serum proteins from benign, non-aggressive and aggressive PTC cases were probed with α-clusterin 78E mouse monoclonal or rabbit polyclonal (ab92548; Abcam) antibody. Both antibodies recognized the expected 40-kDa clusterin protein and showed reduction in serum clusterin in thyroid cancer cases, particularly for aggressive PTC. Band intensities are presented as a proportion of the benign level for the respective blot. Panels C-F show verification of clusterin expression in thyroid tissues by immunohistochemical analysis. Sections from non-aggressive PTC (C), metastatic PTC (D), and normal (E) cases were probed with α-clusterin mouse monoclonal antibody. Increased cytoplasmic staining was observed from normal levels for both thyroid cancers. Furthermore, this increase was more prominent in the aggressive PTC tissue. In all cases, no detectable membrane or nuclear expression of clusterin was observed. Minimal non-specific staining was observed when sections were probed with the 2° antibody alone (F). Original magnification ×400. Panels G and H show secreted serum clusterin levels in thyroid cancer patients using ELISA. Serum samples from 39 cases (10 benign, 10 non-aggressive PTC, 19 aggressive PTC) were examined using the Human Clusterin ELISA kit (BioVendor), as recommended. Bar (G) and scatter (H) plots were generated, depicting mean and standard error values. t-test analyses indicated that a significant difference was observed in serum clusterin levels in benign and aggressive thyroid cancers (p=0.011).

FIG. 5, panels A-D show (ALCAM)/CD166 expression in human thyroid cancer tissue using immunohistochemical analysis. (A) Moderate membrane staining is observed in benign tissue. (B) Intense membrane and cytoplasmic staining in shown for stage I PTC, compared to moderate cytoplasmic staining in stage IV PTC (D). Very weak cytoplasmic staining is shown in ATC (C). Original magnification X400. Panel E shows scatter plot analysis of (ALCAM)/CD166 cytoplasmic and membrane expression. Tissue sections from benign (n=9), non-aggressive (n=16), aggressive (n=19) PTC and ATC (n=5) cases were probed with (ALCAM)/CD166 antibody. The distribution of all immunostaining scores for cytoplasmic and membrane (ALCAM)/CD166 expression is shown, with respective mean and standard error bars indicated. Decreased membrane staining was observed for all non-aggressive and aggressive PTC and ATC cases as compared to the benign cases. Panel F shows secreted (ALCAM)/CD166 in thyroid cancer patients' sera using ELISA. Pre- and post-surgery blood samples were assessed. Non-Agg refers to non-aggressive stage I or II PTC cases with no extrathyroidal spread. Agg PTC refers to aggressive PTC cases that have lymph node metastasis, extrathyroidal invasion, and multifocality. Very aggressive refers to the more aggressive PTC cases, which are poorly differentiated and/or have distant metastasis. Pre and post-surgery non-aggressive PTC and the very aggressive post-surgery PTC (ALCAM)/CD166 levels were lower than all the other groups. The non-aggressive PTC (ALCAM)/CD166 levels were lower as compared to the other groups.

FIG. 6 shows verification of secreted proteins in thyroid cancer patients' sera. Blots containing immunodepleted serum samples (50 μg/lane) were probed with respective antibodies. AXL receptor tyrosine kinase, pyruvate kinase 3 isoform 1 variant (PKM2), APLP2 and amyloid precursor protein (APP) levels were increased in thyroid cancer patients' sera and correlated with aggressiveness. 14-3-3 zeta and protein phosphatase 2A inhibitor, SET, were detected in the sera of thyroid cancer patients as well.

FIG. 7, panels A-F show APLP2 expression in human thyroid cancer tissues using immunohistochemical analysis. (A) Benign tissues showed moderate nuclear and mild cytoplasmic staining (B) Stage II PTC showed moderate nuclear and cytoplasmic staining. (C) Stage IV tall cell variant PTC showed stronger cytoplasmic staining compared to the other thyroid cancer subtypes, with some mild membrane staining (D) Stage IV PTC revealed mild membrane and cytoplasmic staining compared to moderate cytoplasmic staining in multifocal stage IV PTC (E). (F) ATC tissue showed moderate cytoplasmic APLP2 staining. Original magnification ×400. Panel G shows scatter plot analysis of APLP2 nuclear and cytoplasmic expression. Tissue sections from benign (n=3), PTC (n=7) and ATC (n=1) cases were probed with APLP2 antibody. The distribution of all immunostaining scores for nuclear and cytoplasmic APLP2 expression is shown, with respective mean and standard error bars indicated.

FIG. 8 shows secreted AXL protein in thyroid cancer patient sera using an enzyme-linked immunosorbent assay. Pre- and post-surgery blood samples were assessed. Non-Agg refers to non-aggressive stage I or II PTC cases with no extrathyroidal spread. Agg PTC refers to aggressive PTC cases that have lymph node metastasis, extrathyroidal invasion, and multifocality. Very-Agg refers to the more aggressive PTC cases, which are poorly differentiated and/or have distant metastasis. Lt Post-Non-Agg refers to long-term post-surgery non-aggressive PTC blood samples taken 4-30 years after surgery with patients having no residual cancer. In long-term post-surgery non-aggressive PTC and post-surgery very aggressive PTC, AXL sera levels were lower compared to their respective pre-surgery levels.

FIG. 9 show immunohistochemical analysis of APP, 14-3-3 zeta, AXL, SET, PKM2 and hnRNPK in benign tissues and PTC. Cytoplasmic expressions of APP and 14-3-3 zeta proteins were observed in PTCs, in contrast to the absent cytoplasmic and nuclear staining in benign thyroid tissues. Minimal nuclear expression and no cytoplasmic expression of AXL were observed in benign thyroid tissues. Cytoplasmic accumulation of AXL protein was found in the PTC cases examined. Strong nuclear expression and no cytoplasmic expression were observed for SET in benign thyroid tissues. In PTC, SET was found to show reduced nuclear expression. PKM2 displayed nuclear and cytoplasmic expression in benign thyroid tissues. In PTC, cytoplasmic accumulation of the PKM2 protein was found to increase, often with a loss of nuclear expression. hnRNPK was found in the nucleus of benign thyroid tissues. This nuclear expression was found to decrease, with a concomitant increase in cytoplasmic hnRNPK in the PTC cases observed. Original magnification X400.

FIG. 10 shows scatter plot analysis of APP, SET, AXL, 14-3-3 zeta, PKM2 and hnRNPK proteins expression. Tissue sections from benign (n=13) and papillary thyroid carcinomas (PTC, n=13) cases were probed with respective antibodies. The distribution of all immunostaining scores for these proteins expression is shown, with respective mean and standard error bars indicated. Increased cytoplasmic staining of APP (A) and 14-3-3 zeta (D) was observed in PTC cases as compared to benign. Nuclear staining of SET protein was observed in PTC and benign cases (B). Minimal nuclear expression and no cytoplasmic expression of AXL were observed in benign thyroid tissues (C). Cytoplasmic accumulation of AXL protein was found in the PTC cases examined (C). PKM2 displayed nuclear and cytoplasmic expression in benign thyroid tissues (E). In PTC, cytoplasmic accumulation of PKM2 protein was found to increase, often with a loss of nuclear expression (E) hnRNPK was found in the nucleus of benign thyroid tissues (F). This nuclear expression was found to decrease, with a concomitant increase in cytoplasmic hnRNPK in the PTC cases observed (F).

FIG. 11 shows immunohistochemical analysis of APP, AXL, PKM2 and SET proteins in mouse xenografts of human PTC-derived (BCPAP) and ATC-derived (C643) thyroid carcinoma cell lines. In agreement with the observations in human PTC, APP was observed to stain in the cytoplasm of PTC xenografts. Increased cytoplasmic expression of APP was found in the ATC xenografts. Weak cytoplasmic expression of AXL was observed in ATC xenografts. In anaplastic mouse xenografts, decreased nuclear expression of PKM2 in a majority of cells was observed, along with cytoplasmic immunostaining compared to papillary mouse xenografts. Consistent with the observations in human tissues, strong SET nuclear expression was observed in papillary mouse xenografts. Decreased nuclear staining of SET was observed in ATC xenografts, along with cytoplasmic expression of the protein. Original magnification X400.

### DETAILED DESCRIPTION OF THE INVENTION

The invention relates to newly discovered correlations between expression of Thyroid Cancer Markers and thyroid cancer, in particular stage, aggressiveness or metastatic potential of a thyroid cancer. The Thyroid Cancer Markers described herein provide methods for diagnosing, detecting, predicting, monitoring or characterizing thyroid cancer, in particular stage, aggressiveness or metastatic potential of a thyroid cancer. Methods are provided for screening for, diagnosing or detecting the presence or absence of thyroid cancer, papillary, follicular or aggressive or metastatic thyroid cancer in a sample, and for monitoring the progression of thyroid cancer, as well as providing information about characteristics of a thyroid carcinoma that are relevant to the diagnosis and characterization of thyroid carcinoma in a patient.

### GLOSSARY

Unless defined otherwise, all technical and scientific terms used herein have the same meaning as commonly understood by one of ordinary skill in the art to which this invention belongs. The following definitions supplement those in the art and are directed to the present application and are not to be imputed to any related or unrelated case. Although any methods and materials similar or equivalent to those described herein can be used in the practice of the invention, particular materials and methods are described herein.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. As used herein and in the appended claims, the singular forms “a”, “an”, and “the” include plural reference unless the context clearly dictates otherwise.

The term “thyroid cancer” refers to any malignant process of the thyroid gland. Examples of thyroid cancers include, but are not limited to, papillary thyroid carcinoma, follicular variant of papillary thyroid carcinoma, follicular carcinoma, Hurthle cell tumor, anaplastic thyroid carcinoma, medullary thyroid cancer, thyroid lymphoma, poorly differentiated thyroid cancer and thyroid angiosarcoma. In aspects of the invention, the thyroid cancer is papillary thyroid carcinoma. In aspects of the invention, the thyroid cancer is a follicular variant of papillary thyroid carcinoma or follicular carcinoma (also referred to herein as “follicular thyroid cancer”). In aspects of the invention, the thyroid cancer is medullary thyroid cancer. In aspects of the invention, the thyroid cancer is an aggressive cancer or has metastatic potential, in particular an aggressive medullary or follicular thyroid cancer or a medullary or follicular thyroid cancer with metastatic potential. In aspects of the invention, the thyroid cancer is anaplastic thyroid carcinoma (ATC).

“Metastatic potential” refers to the ability or possibility of a cancer cell moving from the initial site (i.e. thyroid) to other sites in the body.

The term “detect” or “detecting” includes assaying, or otherwise screening or establishing the presence or absence of the target marker(s), subunits, or combinations of reagent bound targets, and the like, or assaying for ascertaining, establishing, classifying monitoring, predicting or otherwise determining one or more factual characteristics of a thyroid cancer such as aggressiveness, metastatic potential or patient survival, or assisting in same. A standard may correspond to levels quantitated for samples from control subjects with no disease or early stage disease (e.g., low grade thyroid cancer such as papillary thyroid cancer) or from other samples of the subject.

The term “sample” and the like mean a material known or suspected of expressing or containing Thyroid Cancer Markers, or binding agents such as antibodies specific for Polypeptide Thyroid Cancer Markers. The sample may be derived from a biological source (“biological sample”), such as tissues, extracts, or cell cultures, including cells (e.g. tumor cells), cell lysates, and biological or physiological fluids, such as, for example, whole blood, plasma, serum, saliva, cerebral spinal fluid, sweat, urine, milk, peritoneal fluid and the like. A sample may be used directly as obtained from the source or following a pretreatment to modify the character of the sample, such as preparing plasma from blood, diluting viscous fluids, and the like. In certain aspects of the invention, the sample is a fluid sample. In certain aspects of the invention the sample is serum, plasma, whole blood, urine or saliva. In certain particular aspects of the invention the sample is serum. In certain aspects of the invention, the sample is a human physiological fluid, such as human serum. In aspects of the invention, the sample comprises cells (or nuclei obtained from the cells) from different sites of a tumor.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). As will be appreciated by those skilled in the art, the target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA). (i.e., cRNA; see, for example., Linsley & Schelter, U.S. patent application Ser. No. 09/411,074, or U.S. Pat. Nos. 5,545,522, 5,891,636 or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Moelcular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+ RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemiluminescent label, radiolabel, and colorimetric label.

Target polynucleotides from a patient sample can be labeled differentially from polynucleotides of a standard. The standard can comprise target polynucleotides from normal individuals (e.g. those not afflicted with or pre-disposed to thyroid cancer, in particular pooled from samples from normal individuals or patients with a different disease stage). The target polynucleotides can be derived from the same individual, but taken at different time points, and thus indicate the efficacy of a treatment by a change in expression of the markers, or lack thereof, during and after the course of treatment.

The terms “subject”, “patient” and “individual” are used interchangeably herein and refer to a warm-blooded animal such as a mammal that is afflicted with thyroid cancer, or suspected of having thyroid cancer, being pre-disposed to thyroid cancer, being screened for thyroid cancer or at risk for thyroid cancer. The term includes but is not limited to domestic animals, sports animals, primates and humans. Preferably, the terms refer to a human.

As used herein, the term subject “suspected of having thyroid cancer” refers to a subject that presents one or more symptoms indicative of a thyroid cancer (e.g., a noticeable lump or mass) or is being screened for a cancer (e.g., during a routine physical). A subject suspected of having thyroid carcinoma may also have one or more risk factors. A subject suspected of having thyroid cancer has generally not been tested for cancer. However, a “subject suspected of having thyroid cancer” encompasses an individual who has received an initial diagnosis but for whom the stage of cancer is not known. The term further includes people who once had cancer (e.g., an individual in remission). As used herein, the term subject “at risk for thyroid cancer” refers to a subject with one or more risk factors for developing thyroid carcinoma. Risk factors include, but are not limited to, gender, age, genetic predisposition, environmental exposure, previous incidents of cancer, preexisting non-cancer diseases, and lifestyle. As used herein, the term “characterizing thyroid cancer in a subject” refers to the identification of one or more properties of a cancer sample in a subject, including but not limited to the subject's prognosis or survival. Cancers may be characterized by the identification of the expression of one or more markers, including but not limited to, the Thyroid Cancer Markers disclosed herein.

“Thyroid Cancer Markers for papillary thyroid cancer set out in Table 1” include the markers identified by “*” in Table 1, namely markers chosen from the markers numbered 4, 12, 25 and 26, and polynucleotides encoding same.

“Thyroid Cancer Markers for follicular thyroid cancer set out in Table 1” include the markers identified by “**” in Table 1, namely markers chosen from the markers numbered 21, 22 and 28, and polynucleotides encoding same.

“Thyroid Cancer Markers for aggressive/metastatic thyroid cancer set out in Table 1” include the markers identified by “***” in Table 1, namely markers chosen from the markers numbered 15, 17, 29, 30, 31, 32, 33, 35, 36, 38, 39, 40, 42, 43, 45, 46, 47, 49-58, 60, 65-75, 77, 78, 80, 81 and 83, 88 and polynucleotides encoding same.

Thyroid Cancer Markers include polypeptide or protein markers including without limitation a native-sequence polypeptide, a polypeptide variant, a chimeric protein or fusion protein, isoforms, complexes, all homologs, fragments, precursors, and modified forms and derivatives of the markers (i.e., Polypeptide Thyroid Cancer Markers).

“Polypeptide” and “protein” are used interchangeably herein and indicate at least one molecular chain of amino acids linked through covalent and/or non-covalent bonds. The terms include peptides, oligopeptides, and proteins, and post-translational modifications of the polypeptides, e.g. glycosylations, acetylations, phosphorylations, and the like. Protein fragments, analogues, mutated or variant proteins, fusion proteins, and the like, are also included within the meaning of the terms.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 10%, 20%, 30%, 40%, 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95%, 97%, or 99% amino acid sequence identity with a native-sequence polypeptide. Particular polypeptide variants have at least 70-80%, 85%, 90%, 95%, 97% or 99% amino acid sequence identity to the sequences identified in Table 1 or 2. Such variants include, for instance, polypeptides wherein one or more amino acid residues are added to, or deleted from, the N- or C-terminus of the full-length or mature sequences of the polypeptide, including variants from other species, but exclude a native-sequence polypeptide. In aspects of the invention variants retain the immunogenic activity of the corresponding native-sequence polypeptide.

Sequence identity of two amino acid sequences or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Polypeptide variants include polypeptides comprising amino acid sequences sufficiently identical to or derived from the amino acid sequence of a native polypeptide which includes fewer amino acids than the full-length polypeptides. A portion or fragment of a polypeptide can be a polypeptide which is for example, 3-5, 8-10, 10, 15, 15-20, 20, 25, 30, 35, 40, 45, 50, 60, 70, 80, 90, 100 or more amino acids in length. Portions or fragments in which regions of a polypeptide are deleted can be prepared by recombinant techniques and can be evaluated for one or more functional activities such as the ability to form antibodies specific for a polypeptide. A portion or fragment of a polypeptide may comprise a domain of the polypeptide, in particular an extracellular domain or intracellular domain.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain, several of which are known in the art.

A naturally occurring allelic variant may contain conservative amino acid substitutions from the native polypeptide sequence or it may contain a substitution of an amino acid from a corresponding position in polypeptide homolog, for example, a murine polypeptide.

A polypeptide disclosed herein includes chimeric or fusion proteins. A “chimeric protein” or “fusion protein” comprises all or part (preferably biologically active) of the polypeptide operably linked to a heterologous polypeptide (i.e., a different polypeptide). Within the fusion protein, the term “operably linked” is intended to indicate that the polypeptide and the heterologous polypeptide are fused in-frame to each other. The heterologous polypeptide can be fused to the N-terminus or C-terminus of the polypeptide. A useful fusion protein is a GST fusion protein in which a polypeptide is fused to the C-terminus of GST sequences. Another example of a fusion protein is an immunoglobulin fusion protein in which all or part of a polypeptide is fused to sequences derived from a member of the immunoglobulin protein family. Chimeric and fusion proteins can be produced by standard recombinant DNA techniques.

Polypeptides used in the methods disclosed herein may be isolated from a variety of sources, such as from human tissue types or from other sources, or prepared by recombinant or synthetic methods, or by any combination of these and similar techniques.

“Polynucleotide” refers to a polymeric form of nucleotides of any length, either ribonucleotides or deoxyribonucleotides. The term includes double- and single-stranded DNA and RNA, modifications such as methylation or capping and unmodified forms of the polynucleotide. The terms “polynucleotide” and “oligonucleotide” are used interchangeably herein. A polynucleotide may, but need not, include additional coding or non-coding sequences, or it may, but need not, be linked to other molecules and/or carrier or support materials. Polynucleotide Thyroid Cancer Markers for use in the methods of the invention may be of any length suitable for a particular method. In certain applications the term refers to antisense nucleic acid molecules (e.g. an mRNA or DNA strand in the reverse orientation to a sense Polynucleotide Thyroid Cancer Markers).

Polynucleotide Thyroid Cancer Markers include polynucleotides encoding Polypeptide Thyroid Cancer Markers, including a native-sequence polypeptide, a polypeptide variant including a portion of a Polypeptide Thyroid Cancer Marker, an isoform, precursor, a chimeric protein, complexes, homologs, fragments, precursors, and modified forms and derivatives of the markers.

Polynucleotides used in the methods of the invention include complementary nucleic acid sequences, and nucleic acids that are substantially identical to these sequences (e.g. at least about 10%, 20%, 30%, 40%, or 45%, preferably 50%, 55%, 60%, 65%, 70%, 75%, 80%, 85%, 90%, 95%, 97%, 98%, or 99% sequence identity).

Polynucleotides also include sequences that differ from a nucleic acid sequence due to degeneracy in the genetic code. As one example, DNA sequence polymorphisms within the nucleotide sequence of a Thyroid Cancer Marker disclosed herein may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides which may be used in the methods disclosed herein include nucleic acids that hybridize under stringent conditions, preferably high stringency conditions to a nucleic acid sequence of a Polynucleotide Thyroid Cancer Marker. Appropriate stringency conditions which promote DNA hybridization are known to those skilled in the art, or can be found in Ausubel et al., (eds) Current Protocols in Molecular Biology, John Wiley & Sons, N.Y. (1989), 6.3.1-6.3.6. Generally, stringent conditions may be selected that are about 5° C. lower than the thermal melting point (Tm) for the specific sequence at a defined ionic strength and pH. The Tm is the temperature (under defined ionic strength, pH, and nucleic acid concentration) at which 50% of the probes complementary to a target sequence hybridize at equilibrium to the target sequence. Generally, stringent conditions will be those in which the salt concentration is less than about 1.0M sodium ion or other salts (e.g. about 0.01 to 1.0M sodium ion) and the temperature is at least about 30° C. for short probes, primers or oligonucleotides (e.g. 10-50 nucleotides) and at least 60° C. for longer probes, primers and oligonucleotides. For example, a hybridization may be conducted at 6.0× sodium chloride/sodium citrate (SSC) at about 45° C., followed by a wash of 2.0×SSC at 50° C., or at 42° C. in a solution containing 6×SCC, 0.5% SDS and 50% formamide followed by washing in a solution of 0.1×SCC and 0.5% SDS at 68° C.

Polynucleotide Thyroid Cancer Markers also include truncated nucleic acids or nucleic acid fragments and variant forms of the nucleic acids disclosed or referenced herein that arise by alternative splicing of an mRNA corresponding to a DNA. A fragment of a polynucleotide includes a polynucleotide sequence that comprises a contiguous sequence of approximately at least about 6 nucleotides, in particular at least about 8 nucleotides, more particularly at least about 10-12 nucleotides, and even more particularly 15-20 nucleotides that correspond to (i.e. identical or complementary to), a region of the specified nucleotide sequence.

“Significantly different” levels of markers or a “significant difference” in marker levels in a patient sample compared to a control or standard (e.g. normal levels, levels from a different disease stage, or levels in other samples from a patient) may represent levels that are higher or lower than the standard error of the detection assay, preferably the levels are at least about 1.5, 2, 3, 4, 5, 6, 7, 8, 9 or 10 times higher or lower, respectively, than the control or standard.

“Microarray” and “array,” refer to nucleic acid or nucleotide arrays or protein or peptide arrays that can be used to detect biomolecules associated with thyroid cancer, for instance to measure gene expression. A variety of arrays are available commercially, such as, for example, as the in situ synthesized oligonucleotide array GeneChip™ made by Affymetrix, Inc. or the spotted cDNA array, LifeArray™ made by Incyte Genomics Inc.

“Binding agent” refers to a substance such as a polypeptide, antibody, ribosome, or aptamer that specifically binds to a Polypeptide Thyroid Cancer Marker. A binding agent, in particular an antibody, that “specifically binds” or “binds” (used interchangeably herein) to a target or an antigen or epitope is a term well understood in the art, and methods to determine specific binding are also well known in the art. A binding agent “specifically binds” to a target if it binds with greater affinity, avidity, more readily, and/or with greater duration than it binds to other substances. It will be appreciated that an antibody that specifically binds to a first target may or may not specifically or preferentially bind to a second target. Thus, “specific binding” does not necessarily require (although it can include) exclusive binding but generally refers to preferential binding. Binding properties may be assessed using an ELISA, which may be readily performed by those skilled in the art (see for example, Newton et al, Develop. Dynamics 197: 1-13, 1993). In an embodiment of the invention, antibodies are reactive against a polypeptide marker if they bind with a Kₐ of greater than or equal to 10⁻⁷ M.

A binding agent may be a ribosome, with or without a peptide component, a RNA or DNA molecule, or a polypeptide. A binding agent may be a polypeptide that comprises a Polypeptide Thyroid Cancer Marker sequence, a peptide variant thereof, or a non-peptide mimetic of such a sequence. By way of example a Polypeptide Thyroid Cancer Marker sequence may be a peptide portion of the polypeptide that is capable of modulating a function mediated by the polypeptide.

An aptamer includes a DNA or RNA molecule that binds to nucleic acids and proteins. An aptamer that binds to a Thyroid Cancer Marker can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to synthetic antibodies, monoclonal antibodies, polyclonal antibodies, recombinant antibodies, antibody fragments (such as Fab, Fab′, F(ab′)2), dAb (domain antibody; see Ward, et al, 1989, Nature, 341:544-546), antibody heavy chains, intrabodies, humanized antibodies, human antibodies, antibody light chains, single chain Fᵥₛ (scFv) (e.g., including monospecific, bispecific etc), anti-idiotypic (ant-Id) antibodies, proteins comprising an antibody portion, chimeric antibodies (for example, antibodies which contain the binding specificity of murine antibodies, but in which the remaining portions are of human origin), derivatives, such as enzyme conjugates or labeled derivatives, diabodies, linear antibodies, disulfide-linked Fvs (sdFv), multispecific antibodies (e.g., bispecific antibodies), epitope-binding fragments of any of the above, and any other modified configuration of an immunoglobulin molecule that comprises an antigen recognition site of the required specificity. An antibody includes an antibody of any type (e.g. IgA, IgD, IgE, IgG, IgM and IgY), any class (e.g. IgG1, IgG2, IgG3, IgG4, IgA1 and IgA2), or any subclass (e.g. IgG2a and IgG2b), and the antibody need not be of any particular type, class or subclass. In certain embodiments of the invention the antibodies are IgG antibodies or a class or subclass thereof. An antibody may be from any animal origin including birds and mammals (e.g. human, murine, donkey, sheep, rabbit, goat, guinea pig, camel, horse, or chicken).

A “recombinant antibody” includes antibodies that are prepared, expressed, created or isolated by recombinant means, such as antibodies expressed using a recombinant expression vector transfected into a host cell, antibodies isolated from recombinant, combinatorial antibody libraries, antibodies isolated from an animal (e.g. a mouse or cow) that is transgenic and/or transchromosomal for human immunoglobin genes, or antibodies prepared, expressed, created or isolated by any other means that involves slicing of immunoglobulin gene sequences to other DNA sequences.

A “monoclonal antibody” refers to an antibody obtained from a population of homogenous or substantially homogenous antibodies. Generally each monoclonal antibody recognizes a single epitope on an antigen. In aspects of the invention, a monoclonal antibody is an antibody produced by a single hybridoma or other cell, and it specifically binds to only a Thyroid Cancer Marker as determined, for example by ELISA or other antigen-binding or competitive binding assay known in the art. The term is not limited to a particular method for making the antibody and for example they may be produced by the hybridoma method or isolated from phage libraries using methods known in the art.

Antibodies, including monoclonal and polyclonal antibodies, fragments and chimeras, may be prepared using methods well known to those skilled in the art. Isolated native or recombinant polypeptides may be utilized to prepare antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for polypeptide markers may also be obtained from scientific or commercial sources.

The “status” of a marker refers to the presence, absence or extent/level of the marker or some physical, chemical or genetic characteristic of the marker. Such characteristics include without limitation, expression level, activity level, structure (sequence information), copy number, post-translational modification etc. The status of a marker may be directly or indirectly determined. In some embodiments status is determined by determining the level of a marker in the sample. The “level” of an element in a sample has its conventional meaning in the art, and includes quantitative determinations (e.g. mg/mL, fold change, etc) and qualitative determinations (e.g. determining the presence or absence of a marker or determining whether the level of the marker is high, low or even present relative to a standard).

The term “abnormal status” means that a marker's status in a sample is different from a reference status for the marker. A reference status may be the status of the marker in samples from normal subjects, averaged samples from subjects with the condition or sample(s) from the same subject taken at different times. An abnormal status includes an elevated, decreased, present or absent marker(s). Determining the level of a marker in a sample may include determining the level of the marker in a sample and abnormal status could be either lower levels (including undetectable levels) or higher levels (including any amount over zero) compared to a standard. A subject may have an increased likelihood of a condition disclosed herein if the status of a marker in the subject's sample is correlated with the condition (e.g. a level of the marker is closer to a standard or reference or is present in levels that exceed some threshold value where exceeding that value is correlated with the condition). A subject with an increased likelihood of a condition disclosed herein includes a subject with an abnormal status for a marker and as such the subject has a higher likelihood of the condition than if the subject did not have that status.

An “elevated status” means one or more characteristics of a marker are higher than a standard. In aspects of the invention, the term refers to an increase in a characteristic as compared to a standard. A “low status” means one or more characteristics of a marker are lower than a standard. In aspects of the invention, the term refers to a decrease in a characteristic as compared to a standard. A “negative status” means that one or more characteristic of a marker is absent or undetectable.

### General Methods

A variety of methods can be employed for the diagnostic and prognostic evaluation of thyroid cancer involving Thyroid Cancer Markers and the identification of subjects with a predisposition to such disorders. Such methods may, for example, utilize Polynucleotide Thyroid Cancer Markers and fragments thereof, and binding agents (e.g. antibodies) directed against Polypeptide Thyroid Cancer Markers including peptide fragments. In particular, the polynucleotides and antibodies may be used, for example, for (1) the detection of the presence of polynucleotide mutations, or the detection of either over- or under-expression of mRNA, relative to a non-disorder state or the qualitative or quantitative detection of alternatively spliced forms of polynucleotide transcripts which may correlate with certain conditions or susceptibility toward such conditions; and (2) the detection of either an over- or an under-abundance of polypeptides relative to a non-disorder state or the presence of a modified (e.g., less than full length) polypeptide which correlates with a disorder state, or a progression toward a disorder state.

The methods described herein may be used to evaluate the probability of the presence of malignant cells, for example, in a group of cells freshly removed from a host. Such methods can be used to detect tumors, quantitate and monitor their growth, and help in the diagnosis and prognosis of disease. For example, significantly different levels of one or more markers in Table 1 are indicative of thyroid cancer.

The methods of the invention require that the amount of Thyroid Cancer Markers quantitated in a sample from a subject being tested be compared to a predetermined standard or cut-off value. A standard may correspond to levels quantitated for another sample or an earlier sample from the subject, or levels quantitated for a control sample, in particular a sample from a subject with a lower grade cancer. Levels for control samples from healthy subjects or cancer subjects may be established by prospective and/or retrospective statistical studies. Healthy subjects who have no clinically evident disease or abnormalities may be selected for statistical studies. Diagnosis may be made by a finding of statistically different levels of Thyroid Cancer Markers compared to a control sample or previous levels quantitated for the same subject.

The invention also contemplates the methods described herein using multiple markers for thyroid cancer. Therefore, the invention contemplates a method for analyzing a biological sample for the presence of Thyroid Cancer Markers and other markers that are specific indicators of thyroid cancer. The methods described herein may be modified by including reagents to detect the markers or polynucleotides encoding the markers. Examples of other markers include without limitation the markers listed in Table 2 or markers comprising or selected from the group comprising galectin-3, thyroglobulin, E-cadherin, and galectin-3, in particular galectin-3.

### Nucleic Acid Methods

As noted herein thyroid cancer, in particular the stage or aggressiveness of a thyroid cancer, may be detected based on the level of Polynucleotide Thyroid Cancer Markers in a sample. Techniques for detecting nucleic acid molecules such as polymerase chain reaction (PCR) and hybridization assays are well known in the art.

Probes may be used in hybridization techniques to detect polynucleotides. The technique generally involves contacting and incubating nucleic acids obtained from a sample from a patient or other cellular source with a probe under conditions favorable for the specific annealing of the probes to complementary sequences in the nucleic acids (e.g. under stringent conditions as discussed herein). After incubation, the non-annealed nucleic acids are removed, and the presence of nucleic acids that have hybridized to the probe if any are detected. Nucleotide probes for use in the detection of polynucleotide sequences in samples may be constructed using conventional methods known in the art. The probes may comprise DNA or DNA mimics corresponding to a portion of an organism's genome, or complementary RNA or RNA mimics. The nucleic acids can be modified at the base moiety, at the sugar moiety, or at the phosphate backbone. DNA can be obtained using standard methods such as polymerase chain reaction (PCR) amplification of genomic DNA or cloned sequences. Computer programs known in the art can be used to design primers with the required specificity and optimal amplification properties.

A nucleotide probe may be labeled with a detectable substance such as a radioactive label which provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleic acids to be detected and the amount of nucleic acids available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect Polynucleotide Thyroid Cancer Markers, preferably in human cells. The nucleotide probes may also be useful in the diagnosis of thyroid cancer, involving Polynucleotide Thyroid Cancer Markers in monitoring the progression of thyroid cancer, or monitoring a therapeutic treatment.

The detection of polynucleotides in a sample may involve the amplification of specific gene sequences using an amplification method such as PCR, followed by the analysis of the amplified molecules using techniques known to those skilled in the art. By way of example, oligonucleotide primers may be employed in a PCR based assay to amplify a portion of a polynucleotide and to amplify a portion of a polynucleotide derived from a sample, wherein the oligonucleotide primers are specific for (i.e. hybridize to) the polynucleotides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of a Polynucleotide Thyroid Cancer Marker; that is, they are at least 10 nucleotides, and preferably at least 20 nucleotides in length. In an embodiment the primers and probes are at least about 10-40 nucleotides in length.

Hybridization and amplification reactions may also be conducted under stringent conditions as discussed herein. Hybridization and amplification techniques described herein may be used to assay qualitative and quantitative aspects of polynucleotide expression. For example, RNA may be isolated from a cell type or tissue known to express Polynucleotide Thyroid Cancer Markers, and tested utilizing the hybridization (e.g. standard Northern analyses) or PCR techniques.

In an aspect of the invention, a method is provided employing reverse transcriptase-polymerase chain reaction (RT-PCR), in which PCR is applied in combination with reverse transcription. Generally, RNA is extracted from a sample using standard techniques and is reverse transcribed to produce cDNA. The cDNA is used as a template for a polymerase chain reaction. The cDNA is hybridized to primer sets which are specifically designed against a Polynucleotide Thyroid Cancer Marker. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected thyroid cancer, an individual who is not afflicted with thyroid cancer or has early stage disease or has aggressive or metastatic disease. The reaction may be performed on several dilutions of cDNA spanning at least two orders of magnitude. A statistically significant difference in expression in several dilutions of the subject sample as compared to the same dilutions of the non-cancerous sample or early-stage cancer sample may be considered positive for the presence of cancer.

Oligonucleotides or longer fragments derived from Polynucleotide Thyroid Cancer Markers may be used as targets in a microarray. The microarray can be used to monitor the expression levels of the polynucleotides and to identify genetic variants, mutations, and polymorphisms. The information from the microarray may be used to determine gene function, to understand the genetic basis of a disorder, to diagnose a disorder, and to develop and monitor the activities of therapeutic agents. Thus, the invention also includes an array comprising Polynucleotide Thyroid Cancer Markers, and optionally other thyroid cancer markers. The array can be used to assay expression of Polynucleotide Thyroid Cancer Markers in the array. The invention allows the quantitation of expression of the polynucleotides.

The invention provides microarrays comprising Polynucleotide Thyroid Cancer Markers. In one embodiment, the invention provides a microarray for distinguishing samples associated with thyroid cancer, in particular aggressive thyroid cancer or thyroid cancer with metastatic potential comprising a positionally-addressable array of polynucleotide probes bound to a support, the polynucleotide probes comprising sequences complementary and hybridizable to Polynucleotide Thyroid Cancer Markers. In an embodiment, the array can be used to monitor the time course of expression of Polynucleotide Thyroid Cancer Markers in the array. This can occur in various biological contexts such as tumor progression. An array can also be useful for ascertaining differential expression patterns of Polynucleotide Thyroid Cancer Markers, and optionally other thyroid cancer markers in normal and abnormal cells. This may provide a battery of nucleic acids that could serve as molecular targets for diagnosis or therapeutic intervention. The preparation, use, and analysis of microarrays are well known to those skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662)

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, NY, 1988). In general, a method of the invention may comprise (a) contacting a sample from the subject with a binding agent; (b) detecting in the sample a level of polypeptide that binds to the binding agent; and (c) comparing the level of polypeptide with a predetermined standard or cut-off value. In particular aspects of the invention, the binding agent is an antibody.

In an aspect, the invention provides a diagnostic method for monitoring or diagnosing thyroid cancer in a subject by quantitating Polypeptide Thyroid Cancer Markers in a biological sample from the subject comprising reacting the sample with antibodies specific for Polypeptide Thyroid Cancer Markers which are directly or indirectly labeled with detectable substances and detecting the detectable substances.

In an aspect of the invention, a method for detecting or diagnosing thyroid cancer is provided comprising or consisting essentially of: (a) obtaining a sample suspected of containing Polypeptide Thyroid Cancer Markers; (b) contacting said sample with antibodies that specifically bind Polypeptide Thyroid Cancer Markers under conditions effective to bind the antibodies and form complexes; (c) measuring the amount of Polypeptide Thyroid Cancer Markers present in the sample by quantitating the amount of the complexes; and (d) comparing the amount of Polypeptide Thyroid Cancer Markers present in the samples with the amount of Polypeptide Thyroid Cancer Markers in a control, wherein a change or significant difference in the amount of Polypeptide Thyroid Cancer Markers in the sample compared with the amount in the control is indicative of thyroid cancer, stage of thyroid cancer, progression, aggressiveness and/or metastatic potential of the thyroid cancer.

In an embodiment, the invention contemplates a method for monitoring the progression of thyroid cancer in an individual, comprising: (a) contacting antibodies which bind to Polypeptide Thyroid Cancer Markers with a sample from the individual so as to form complexes comprising the antibodies and Polypeptide Thyroid Cancer Markers in the sample; (b) determining or detecting the presence or amount of complex formation in the sample; (c) repeating steps (a) and (b) at a point later in time; and (d) comparing the result of step (b) with the result of step (c), wherein a difference in the amount of complex formation is indicative of disease, disease stage, progression, aggressiveness and/or metastatic potential of the cancer in said individual. The amount of complexes may also be compared to a value representative of the amount of the complexes from an individual not at risk of, or afflicted with thyroid cancer at a different stage.

Antibodies specifically reactive with Polypeptide Thyroid Cancer Markers or derivatives, such as enzyme conjugates or labeled derivatives, may be used to detect Polypeptide Thyroid Cancer Markers in various samples (e.g. biological materials, in particular tissue samples). They may be used as diagnostic or prognostic reagents and they may be used to detect abnormalities in the level of Polypeptide Thyroid Cancer Markers or abnormalities in the structure, and/or temporal, tissue, cellular, or subcellular location of Polypeptide Thyroid Cancer Markers. Antibodies may also be used to screen potentially therapeutic compounds in vitro to determine their effects on thyroid cancer involving Polypeptide Thyroid Cancer Markers. In vitro immunoassays may also be used to assess or monitor the efficacy of particular therapies.

Antibodies may be used in any immunoassay that relies on the binding interaction between antigenic determinants of Polypeptide Thyroid Cancer Markers and the antibodies. Immunoassay procedures for in vitro detection of antigens in samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of Polypeptide Thyroid Cancer Markers in a sample may be accomplished by competitive or non-competitive immunoassay procedures in either a direct or indirect format. Detection of Polypeptide Thyroid Cancer Markers using antibodies can, for example involve immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. Alternatively, the binding of antibodies to Polypeptide Thyroid Cancer Markers can be detected directly using, for example, a surface plasmon resonance (SPR) procedure such as, for example, Biacore®, microcalorimetry or nano-cantilivers. These terms are well understood by those skilled in the art, and they will know, or can readily discern, other immunoassay formats without undue experimentation.

Antibodies specific for Polypeptide Thyroid Cancer Markers may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels, (e.g., FITC, rhodamine, lanthanide phosphors), luminescent labels such as luminol; and enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labelled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA. A bioluminescent compound may also be used as a detectable substance. Examples of bioluminescent detectable substances are luciferin, luciferase and aequorin.

Indirect methods may also be employed in which the primary antigen-antibody reaction is amplified by the introduction of a second antibody, having specificity for the antibody reactive against Polypeptide Thyroid Cancer Markers. By way of example, if the antibody having specificity against Polypeptide Thyroid Cancer Markers is a rabbit IgG antibody, the second antibody may be goat anti-rabbit IgG, Fc fragment specific antibody labeled with a detectable substance as described herein.

Methods for conjugating or labelling the antibodies discussed above may be readily accomplished by one of ordinary skill in the art.

Cytochemical techniques known in the art for localizing antigens using light and electron microscopy may be used to detect Polypeptide Thyroid Cancer Markers. Generally, an antibody may be labeled with a detectable substance and a Polypeptide Thyroid Cancer Marker may be localized in tissues and cells based upon the presence of the detectable substance.

In the context of the methods of the invention, the sample, binding agents (e.g. antibodies), or Polypeptide Thyroid Cancer Markers may be immobilized on a carrier or support, such as, for example, agarose, cellulose, nitrocellulose, dextran, Sephadex, Sepharose, liposomes, carboxymethyl cellulose, polyacrylamides, polystyrene, filter paper, ion-exchange resin, plastic film, nylon or silk. The support material may have any possible configuration including spherical cylindrical or flat. Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for Polypeptide Thyroid Cancer Markers may be immobilized using sheep anti-mouse IgG Fc fragment specific antibody coated on the carrier or support.

Where a radioactive label is used as a detectable substance, a Polypeptide Thyroid Cancer Marker may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T K and Diamandis E P Anal. Chem., 1992:64:342-346 may be used with a conventional time-resolved fluorometer.

According to an embodiment of the invention, an immunoassay for detecting Polypeptide Thyroid Cancer Markers in a biological sample comprises contacting an amount of a binding agent that specifically binds to Polypeptide Thyroid Cancer Markers in the sample under conditions that allow the formation of a complex(es) comprising the binding agent and Polypeptide Thyroid Cancer Markers and determining the presence or amount of the complex(es) as a measure of the amount of the Polypeptide Thyroid Cancer Markers contained in the sample.

In accordance with an embodiment of the invention, a method is provided wherein Polypeptide Thyroid Cancer Markers antibodies are directly or indirectly labelled with enzymes, substrates for the enzymes are added wherein the substrates are selected so that the substrates, or a reaction product of an enzyme and substrate, form fluorescent complexes with lanthanide metals, preferably europium and terbium. A lanthanide metal(s) is added and Polypeptide Thyroid Cancer Markers are quantitated in the sample by measuring fluorescence of the fluorescent complexes. Enzymes are selected based on the ability of a substrate of the enzyme, or a reaction product of the enzyme and substrate, to complex with lanthanide metals. Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer.

Antibodies specific for Polypeptide Thyroid Cancer Markers may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In embodiments, antibodies specific for Polypeptide Thyroid Cancer Markers are labelled with enzymes.

Aspects of the methods of the invention involve (a) reacting a biological sample from a subject with antibodies specific for Polypeptide Thyroid Cancer Markers wherein the antibodies are directly or indirectly labelled with enzymes; (b) adding substrates for the enzymes wherein the substrates are selected so that the substrates, or reaction products of the enzymes and substrates form fluorescent complexes; (c) quantitating Polypeptide Thyroid Cancer Markers in the sample by measuring fluorescence of the fluorescent complexes; and (d) comparing the quantitated levels to levels obtained for other samples from the subject patient, or control subjects. In an embodiment, the Polypeptide Thyroid Cancer Markers are set out in Table 1 and the quantitated levels are compared to levels quantitated for normal subjects or subjects with an early stage of disease wherein a significant difference in the levels of the markers compared with the control subjects is indicative of thyroid cancer, or stage or aggressiveness of thyroid cancer.

A particular embodiment of the invention comprises the following steps: (a) incubating a biological sample with a first antibody specific for Polypeptide Thyroid Cancer Markers which is directly or indirectly labeled with a detectable substance, and a second antibody specific for Polypeptide Thyroid Cancer Markers which is immobilized; (b) separating the first antibody from the second antibody to provide a first antibody phase and a second antibody phase; (c) detecting the detectable substance in the first or second antibody phase thereby quantitating Polypeptide Thyroid Cancer Markers in the biological sample; and (d) comparing the quantitated Polypeptide Thyroid Cancer Markers with levels for a predetermined standard. The standard may correspond to levels quantitated for samples from control subjects with no disease or early stage disease or from other samples of the subject including earlier samples of the subject.

In accordance with an embodiment, the present invention provides means for determining Polypeptide Thyroid Cancer Markers in a sample by measuring Polypeptide Thyroid Cancer Markers by immunoassay. It will be evident to a skilled artisan that a variety of competitive or non-competitive immunoassay methods can be used to measure Polypeptide Thyroid Cancer Markers in serum. Competitive methods typically employ immobilized or immobilizable antibodies to Polypeptide Thyroid Cancer Markers and labeled forms of Polypeptide Thyroid Cancer Markers. Sample Polypeptide Thyroid Cancer Markers and labeled Polypeptide Thyroid Cancer Markers compete for binding to antibodies specific for Polypeptide Thyroid Cancer Markers. After separation of the resulting labeled Polypeptide Thyroid Cancer Markers that have become bound to antibody (bound fraction) from that which has remained unbound (unbound fraction), the amount of the label in either bound or unbound fraction is measured and may be correlated with the amount of Polypeptide Thyroid Cancer Markers in the test sample in any conventional manner, e.g., by comparison to a standard curve.

In another aspect, a non-competitive method is used for the determination of Polypeptide Thyroid Cancer Markers with the most common method being the “sandwich” method. In this assay, two antibodies specific for a Polypeptide Thyroid Cancer Marker are employed. One of the antibodies is directly or indirectly labeled (the “detection antibody”), and the other is immobilized or immobilizable (the “capture antibody”). The capture and detection antibodies can be contacted simultaneously or sequentially with the test sample. Sequential methods can be accomplished by incubating the capture antibody with the sample, and adding the detection antibody at a predetermined time thereafter or the detection antibody can be incubated with the sample first and then the capture antibody added. After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises Polypeptide Thyroid Cancer Marker “sandwiched” between the capture and detection antibodies. In another embodiment, the label may be measured without separating the capture antibody and liquid test mixture.

In particular sandwich immunoassays of the invention mouse polyclonal/monoclonal antibodies specific for Polypeptide Thyroid Cancer Markers and rabbit polyclonal/monoclonal antibodies specific for Polypeptide Thyroid Cancer Markers are utilized.

In a typical two-site immunometric assay for Polypeptide Thyroid Cancer Markers one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In an aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

The invention also contemplates diagnostic methods employing mass spectrometry. In an aspect, the invention relates to a method for diagnosing or screening for thyroid cancer in a subject comprising: (a) extracting proteins from a sample from the subject and producing a profile of the proteins by subjecting the proteins to mass spectrometry; and (b) comparing the profile with a profile for a reference comprising Thyroid Cancer Marker sets of the invention.

Proteins may be extracted from the samples in a manner known in the art. For example, proteins may be extracted by ultra-centrifugation or other standard techniques. The separated proteins may be digested into peptides, in particular using proteolytic enzymes such as trypsin, pepsin, subtilisin, and proteinase. For example, proteins may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

Mass spectrometers that may be used to analyze the peptides or proteins include a Matrix-Assisted Laser Desorption/Ioniation Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

### Screening Methods

The invention contemplates a method of assessing the potential of a test compound to contribute to thyroid cancer comprising: (a) maintaining separate aliquots of thyroid cancer cells in the presence and absence of the test compound; and (b) comparing the levels of Thyroid Cancer Markers associated with the thyroid cancer in each of the aliquots. A significant difference between the levels of Thyroid Cancer Markers in an aliquot maintained in the presence of (or exposed to) the test compound relative to the aliquot maintained in the absence of the test compound, indicates that the test compound potentially contributes to thyroid cancer.

The invention also contemplates methods for evaluating test agents or compounds for their potential efficacy in treating thyroid cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides (e.g. antisense, siRNA), and small organic or inorganic molecules. The agents or compounds may be endogenous physiological compounds or natural or synthetic compounds.

The invention provides a method for assessing the potential efficacy of a test agent for potential efficacy in treating thyroid cancer in a patient the method comprising comparing: (a) levels of one or more Thyroid Cancer Markers, and optionally other markers of thyroid cancer, in a first sample obtained from a patient and exposed to the test agent; and (b) levels of one or more Thyroid Cancer Markers, and optionally other markers, in a second sample obtained from the patient, wherein the sample is not exposed to the test agent, wherein a significant difference in the levels of expression of one or more Thyroid Cancer Markers, and optionally the other markers, in the first sample, relative to the second sample, is an indication that the test agent is potentially efficacious for treating thyroid cancer in the patient. The first and second samples may be portions of a single sample obtained from a patient or portions of pooled samples obtained from a patient.

In an aspect, the invention provides a method of selecting an agent for treating thyroid cancer, in particular aggressive thyroid cancer in a patient comprising: (a) obtaining a sample from the patient; (b) separately maintaining aliquots of the sample in the presence of a plurality of test agents; (c) comparing one or more Thyroid Cancer Markers, and optionally other markers, in each of the aliquots; (d) selecting one of the test agents which alters the levels of one or more Thyroid Cancer Markers, and optionally other markers in the aliquot containing that test agent, relative to other test agents; and (e) optionally administering the selected test to the patient.

The invention further relates to a method of assessing the efficacy of a therapy for modulating thyroid cancer in a patient. A method of the invention comprises comparing: (a) levels of Thyroid Cancer Markers in a first sample from the patient obtained from the patient prior to providing at least a portion of the therapy to the patient; and (b) levels of Thyroid Cancer Markers in a second sample obtained from the patient following therapy. In an embodiment, a significant difference between the levels of Thyroid Cancer Markers in the second sample relative to the first sample or an abnormal state is an indication that the therapy is efficacious for modulating the thyroid cancer. In a particular embodiment, the method is used to assess the efficacy of a therapy for treating a thyroid cancer where lower levels of Thyroid Cancer Markers in the second sample relative to the first sample, is an indication that the therapy is efficacious. The “therapy” may be any therapy for treating thyroid cancer including but not limited to therapeutics, gene therapy, and surgery. Therefore, the method can be used to evaluate a patient before, during, and after therapy.

The invention contemplates a method for determining the effect of an environmental factor on thyroid cancer comprising comparing Thyroid Cancer Markers in the presence and absence of the environmental factor.

### Kits

The invention contemplates kits for carrying out the methods of the invention to diagnosis thyroid cancer or stage of thyroid cancer, and to detect the aggressiveness or metastatic potential of a thyroid cancer. Such kits typically comprise two or more components required for performing a diagnostic assay. Components include but are not limited to compounds, reagents, containers, and/or equipment. Accordingly, the methods described herein may be performed by utilizing pre-packaged test or diagnostic kits comprising at least agents (e.g. antibodies, probes, primers, etc) described herein, which may be conveniently used, e.g., in clinical settings, to diagnose patients, in particular patients afflicted with thyroid cancer, suspected of having thyroid cancer, or at risk of thyroid cancer or exhibiting a predisposition to developing thyroid cancer, and more particularly to determine the aggressiveness or metastatic potential of a thyroid cancer.

The invention contemplates a kit with a container comprising a binding agent(s) as described herein for characterizing a thyroid cancer. By way of example, the kit may contain antibodies specific for a Polypeptide Thyroid Cancer Marker(s), antibodies against the antibodies labelled with an enzyme(s), and a substrate for the enzyme(s). The kit may also contain microtiter plate wells, standards, assay diluent, wash buffer, adhesive plate covers, and/or instructions for carrying out a method of the invention using the kit.

In an aspect, the invention provides a test kit for diagnosing thyroid cancer in a subject which comprises an antibody that binds to a Polypeptide Thyroid Cancer Marker(s) and/or polynucleotides that hybridize to or amplify Polynucleotide Thyroid Cancer Marker(s). In another aspect the invention relates to use of an antibody that binds to a Polypeptide Thyroid Cancer Marker and/or a polynucleotide that hybridizes to or amplifies a Polynucleotide Thyroid Cancer Marker, in the manufacture of a composition for detecting or characterizing a thyroid cancer.

In a further aspect of the invention, the kit includes antibodies or antibody fragments which bind specifically to epitopes of Polypeptide Thyroid Cancer Marker(s) and means for detecting binding of the antibodies to their epitopes associated with thyroid cancer cells, either as concentrates (including lyophilized compositions), which may be further diluted prior to testing. In particular, the invention provides a kit for diagnosing or characterizing thyroid cancer comprising a known amount of a first binding agent that specifically binds to a Polypeptide Thyroid Cancer Marker(s) wherein the first binding agent comprises a detectable substance, or it binds directly or indirectly to a detectable substance.

A kit may be designed to detect the levels of Polynucleotide Thyroid Cancer Markers in a sample. Such kits generally comprise oligonucleotide probes or primers, as described herein, which hybridize to or amplify Polynucleotide Thyroid Cancer Markers. Oligonucleotides may be used, for example, within PCR or hybridization procedures. Test kits useful for detecting target Polynucleotide Thyroid Cancer Markers are also provided which comprise a container containing a Polynucleotide Thyroid Cancer Marker, and fragments or complements thereof.

The kits of the invention can further comprise containers with tools useful for collecting test samples (e.g. serum) including lancets and absorbent paper or cloth for collecting and stabilizing blood.

### Computer Systems

Analytic methods contemplated herein can be implemented by use of computer systems and methods described below and known in the art. Thus, the invention provides computer readable media comprising one or more Thyroid Cancer Markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more markers disclosed herein.

A variety of data processor programs and formats can be used to store information on one or more Thyroid Cancer Markers. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has thyroid cancer, in particular aggressive thyroid cancer, or a pre-disposition to such condition, comprising determining the presence or absence of one or more Thyroid Cancer Markers, and based on the presence or absence of the markers, determining the condition or a pre-disposition to the condition, optionally recommending a procedure or treatment.

The invention also provides in an electronic system and/or in a network, a method for determining whether a subject has a condition disclosed herein, or a pre-disposition to a condition disclosed herein, comprising determining the presence or absence of one or more markers, and based on the presence or absence of the markers, determining whether the subject has the condition or a pre-disposition to the condition, and optionally recommending a procedure or treatment.

The invention further provides in a network, a method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from the network corresponding to the markers; and (c) based on the phenotypic information and information on the markers, determining whether the subject has the condition or a pre-disposition to the condition, and (d) optionally recommending a procedure or treatment.

The invention still further provides a system for identifying selected records that identify a diseased cell or tissue. A system of the invention generally comprises a digital computer; a database server coupled to the computer; a database coupled to the database server having data stored therein, the data comprising records of data comprising one or more markers disclosed herein, and a code mechanism for applying queries based upon a desired selection criteria to the data file in the database to produce reports of records which match the desired selection criteria.

The invention contemplates a business method for determining whether a subject has a condition disclosed herein or a pre-disposition to a condition disclosed herein comprising: (a) receiving phenotypic information on the subject and information on one or more markers disclosed herein associated with samples from the subject; (b) acquiring information from a network corresponding to the markers; and (c) based on the phenotypic information, information on the markers and acquired information, determining whether the subject has the condition or a pre-disposition to the condition, and optionally recommending a procedure or treatment.

In an aspect of the invention, the computer systems, components, and methods described herein are used to monitor a condition or determine the stage of a condition.

### Therapeutic Applications

The invention contemplates therapeutic applications associated with the Thyroid Cancer Markers disclosed herein including thyroid cancer. Thyroid Cancer Markers may be a target for therapy. For example, markers in Table 1 can be a target for treatment of thyroid cancers.

Therapeutic methods include immunotherapeutic methods including the use of antibody therapy. In one aspect, the invention provides one or more antibodies that may be used to prevent thyroid cancer. In another aspect, the invention provides a method of preventing, inhibiting or reducing thyroid cancer comprising administering to a patient an antibody which binds to a Thyroid Cancer Marker in an amount effective to prevent, inhibit, or reduce the condition or the onset of the condition. The invention also contemplates a method of treating thyroid cancer in a subject, comprising delivering to the subject in need thereof, an antibody specific for a Thyroid Cancer Marker in Table 1, in particular Thyroid Cancer Marker in Table 1 that is upregulated in thyroid cancer or a stage of thyroid cancer. According to one aspect of the invention, there is provided a method of treating a subject having thyroid cancer wherein an antibody specific for a marker in Table 1 is administered in a therapeutically effective amount. In a further aspect, the antibody is provided in a pharmaceutically acceptable form.

An antibody which binds to a Thyroid Cancer Marker may be in combination with a label, drug or cytotoxic agent, a target-binding region of a receptor, an adhesion molecule, a ligand, an enzyme, a cytokine, or a chemokine. In aspects of the invention, the Thyroid Cancer Marker may be conjugated to cytotoxic agents (e.g., chemotherapeutic agents) or toxins or active fragments thereof. Examples of toxins and corresponding fragments thereof include diptheria A chain, exotoxin A chain, ricin A chain, abrin A chain, curcin, crotin, phenomycin, enomycin and the like. A cytotoxic agent may be a radiochemical prepared by conjugating radioisotopes to antibodies, or binding of a radionuclide to a chelating agent that has been covalently attached to the antibody. An antibody may also be conjugated to one or more small molecule toxins, such as a calicheamicin, a maytansine, a trichothene, and CC1065 (see U.S. Pat. No. 5,208,020).

The methods of the invention contemplate the administration of single antibodies as well as combinations, or “cocktails”, of different individual antibodies such as those recognizing different epitopes of other markers. Such cocktails may have certain advantages inasmuch as they contain antibodies that bind to different epitopes of Thyroid Cancer Markers and/or exploit different effector mechanisms. Such antibodies in combination may exhibit synergistic therapeutic effects. In addition, the administration of one or more marker specific antibodies may be combined with other therapeutic agents. The specific antibodies may be administered in their “naked” or unconjugated form, or may have therapeutic agents conjugated to them.

In an aspect, the invention provides a pharmaceutical composition for the treatment of thyroid cancer characterized in that the composition comprises an antibody specific for a marker in Table 1, in particular a Thyroid Cancer Marker that is upregulated in thyroid cancer or a type of thyroid cancer, together with a pharmaceutically acceptable carrier, excipient or vehicle.

Antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

One or more marker specific antibody formulations may be administered via any route capable of delivering the antibodies to the site or injury. Routes of administration include, but are not limited to, intravenous, intraperitoneal, intramuscular, intradermal, and the like.

Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the condition, stage of the condition, the binding affinity and half life of the antibodies used, the degree of marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more marker antibodies is also possible and may have advantages in certain situations.

Patients may be evaluated for Thyroid Cancer Markers in order to assist in the determination of the most effective dosing regimen and related factors. The assay methods described herein, or similar assays, may be used for quantitating marker levels in patients prior to treatment. Such assays may also be used for monitoring throughout therapy, and may be useful to gauge therapeutic success in combination with evaluating other parameters such as levels of markers.

Polynucleotide Thyroid Cancer Markers disclosed herein can be turned off by transfecting a cell or tissue with vectors that express high levels of the polynucleotides. Such constructs can inundate cells with untranslatable sense or antisense sequences. Even in the absence of integration into the DNA, such vectors may continue to transcribe RNA molecules until all copies are disabled by endogenous nucleases. Vectors derived from retroviruses, adenovirus, herpes or vaccinia viruses, or from various bacterial plasmids, may be used to deliver polynucleotides to a targeted organ, tissue, or cell population. Methods well known to those skilled in the art may be used to construct recombinant vectors that will express polynucleotides such as antisense. (See, for example, the techniques described in Sambrook et al (supra) and Ausubel et al (supra).)

Methods for introducing vectors into cells or tissues include those methods known in the art which are suitable for in vivo, in vitro and ex vivo therapy. For example, delivery by transfection or by liposomes is well known in the art.

Modifications of gene expression can be obtained by designing antisense molecules, DNA, RNA or PNA, to the regulatory regions of a Polynucleotide Thyroid Cancer Marker, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of a polynucleotide marker.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

In some aspects one or more Polypeptide Thyroid Cancer Markers and polynucleotides encoding the markers, and fragments thereof, may be used in the treatment of a thyroid cancer in a subject. In an aspect the Thyroid Cancer Marker is down-regulated in thyroid cancer. The markers may be formulated into compositions for administration to subjects suffering from a thyroid cancer. Therefore, the present invention also relates to a composition comprising one or more Thyroid Cancer Markers, preferably a Thyroid Cancer Marker downregulated in thyroid cancer, and a pharmaceutically acceptable carrier, excipient or diluent. A method for treating or preventing a thyroid cancer in a subject is also provided comprising administering to a patient in need thereof, one or more one or more Polypeptide Thyroid Cancer Markers and polynucleotides encoding the markers, or a composition of the invention.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

A composition described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999. On this basis, the compositions include, albeit not exclusively, solutions of the active substances in association with one or more pharmaceutically acceptable vehicles or diluents, and contained in buffered solutions with a suitable pH and iso-osmotic with the physiological fluids.

A composition is indicated as a therapeutic agent either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting examples are illustrative of the present invention:

### Example 1

Proteins that are secreted by cultured cancer cells into the media of their cell culture plates (i.e. “secretome” proteins) make especially appealing targets for study because they may be detectable in bodily fluids. The study described in this example examines the secretome of seven thyroid cancer cell lines: TPC-1, BCPAP, CAL 62, SW1736, C643, MRO, and WRO. Proteomic analysis of the conditioned serum-free media of these cells using LC-MS/MS allows for identification of proteins that these cancer cells secrete. This serves as a surrogate for proteins that human thyroid cancer cells secrete in vivo. Identification of secretome proteins has lead to the discovery of numerous potential thyroid cancer biomarkers that may be used to predict aggressiveness of thyroid cancers. Furthermore, the study independently validates selected secretome proteins in the sera of thyroid cancer patients versus cancer-free individuals using Western blots.

The following materials and methods were employed in the Study described in this Example.

### Materials and Methods

Cell Culture and Serum Free Media Collection. Seven thyroid cancer cell lines are used in this study TPC-1 (papillary), BCPAP (papillary), CAL62 (anaplastic), SW1736 (anaplastic), C643 (anaplastic), MRO (follicular), and WRO (follicular). The cells were grown in 25 mL of conditioned RPMI-1640 cell culture media (containing antibodies and supplemented with 10% fetal bovine serum) in 150 mm dishes to approximately 65% confluence. Cells were kept at 37° C. in a humidified atmosphere of 5% CO₂/95% air. The conditioned media was then aspirated and cells were washed three times with phosphate-buffered saline (PBS). Thereafter, cells were washed once with serum-free media (SFM) that was collected as a time 0 h control. Cells were incubated in the SFM for 48 hours. Following 48 h, the SFM was collected, centrifuged at 2200 RPM for 5 minutes at 4° C., and filtered using a 0.2 μm nylon filter. Upon filtration collected SFM samples were immediately frozen at −80° C. until later processing. SFM was collected from sixty 150 mm plates for TPC-1, SW1736 and CAL 62, and from twenty-five 150 mm BCPAP, C643, WRO and MRO plates.

Protein Precipitation from collected SFM and Preparation for LC-MS/MS analysis. Proteins were isolated from SFM using 0.2% sodium deoxycholate (Sigma Aldrich, MO) and 10% trichloroacetic acid (Sigma Aldrich, MO) as described earlier. [4] Following 2 h incubation on ice, the samples were centrifuged at 11 000 g for 30 minutes and washed two times with ice-cold acetone. The precipitated proteins were then dissolved in 50 mM NaHCO₃ buffer. Protein concentration was later determined using the Bradford assay (Bio-Rad, CA). Protein samples were then heated for 1 h at 65° C. in the presence of 5 mM dithiothreitol, cooled to room temperature, and incubated in the dark for 1 h with 10 mM iodoacetamide to allow for alkylation. Sequencing grade trypsin (Promega, Wis.) at 1:20 (w/w) in 50 mM ammonium bicarbonate was subsequently added and the samples were incubated at 37° C. overnight. Digested samples were then dried under vacuum and redissolved in 10 μL of 0.1% formic acid.

Liquid Chromatography-MS/MS analysis. Samples were analyzed by online LC-MS/MS in triplicates. The nanobore LC system and MS/MS setup was followed and has been described earlier [5]. The liquid chromatograph used in the experiment was an LC Packings Ultimate unit (Amsterdam, The Netherlands). The mass spectrometer used was a QSTAR Pulsar-i hybrid quadrupole/time-of flight (QqTOF) instrument (Applied Biosystems/MDS SCIEX, CA). An autosampler was used to load 1 μL of sample onto a C18 reverse-phase precolumn (LC Packings 300 μm×5 mm). Subsequently, reverse-phase chromatography on an analytical column (75 μm×150 mm packed in-house with 3-μm Kromasil C18 beads with 100 Å pores, The Nest Group) was used. For separation, a nonlinear binary gradient was used: eluant A consisting of 94.9% deionized water, 5.0% acetonitrile, and 0.1% formic acid (pH 3); and eluant B consisting of 5.0% deionized water, 94.9% acetonitrile, and 0.1% formic acid. During the first 5 min of the LC run, eluant A at a flow rate of 25 μL min⁻¹ was used to load peptides from the sample onto the C18 precolumn. Desalting continued for two additional min. At the 8^{th} min, the C18 precolumn was switched inline with the reverse-phase analytical column; separation was performed at 200 mL min⁻¹ using a 180-min binary gradient shown below.

| | Time (min) | | | | | | | | |
|---|---|---|---|---|---|---|---|---|---|
| | 0 | 5 | 10 | 120 | 140 | 145 | 155 | 157 | 189 |
| B (%) | 5 | 5 | 15 | 35 | 60 | 80 | 80 | 5 | Stop |

MS/MS Settings and Data Collection. Data was collected in information-dependent acquisition (IDA) mode using Analyst QS 1.1 and Bioanalyst Extension 1.1 software (Applied Biosystems/MDS SCIEX). MS cycles consisted of a TOF MS survey scan with an m/z range of 400-1500 Th for 1 s. This was followed by five product-ion scans with an m/z range of 80-2000 Th for 2 s each. IDA CE Parameters script was used to control the collision energy (CE). Switching criteria were set to ions with m/z≧400 and ≦1500 Th, charge states of 2-4, and abundances of ≧10 counts. Former target ions were excluded for 30 s, and ions within a 6-Th window were ignored. Additionally, the IDA Extensions II script was set to “no repetition” before dynamic exclusion and to select a precursor ion nearest to a threshold of 10 counts on every fourth cycle. LC-MS/MS data were searched using the ProteinPilot software (Applied Biosystems, Foster City, Calif.) and a Celera human protein database (CDS KBMS 20041109) containing 178239 protein sequences. The cutoff for significance used for this search was set for a score of 1.3, which corresponds to a confidence score of 95%.

Secretion features of identified proteins. To analyze identified proteins' secretion features, Signal Peptide Predictor (SignalP, http://www.cbs.dtu.dk/services/SignalP 3.0) was used. SignalP uses amino-acid sequences to predict the existence and location of signal peptide cleavage sites. SignalP determines the likelihood a protein is a signaling peptide by using numerous artificial neural networks and hidden Markov model algorithms to detect signal peptides from protein sequences. A protein is considered classically secreted if it receives a signal peptide probability ≧0.900. In order to identify non-classical, or leaderless, protein secretion SecretomeP (http://www.cbs.dtu.dk/services/SecretomeP 2.0) was used. SecretomeP uses a neural network that combines six protein characteristics to determine if a protein is non-classically secreted. The protein characteristics include: the number of atoms, number of positively charged residues, presence of transmembrane helices, presence of low-complexity regions, presence of pro-peptides, and subcellular localization. A protein is considered non-classically secreted if it receives an NN-score ≧0.500 (note: only proteins that were not considered classically secreted, i.e. received SignalP scores <0.900, were analyzed using SecretomeP).

Western Blot Verification of Biomarkers in Sera. Western Blots were used to verify the expression of selected secretory proteins, E-cadherin, Nucleolin, CYR61 (cysteine rich angiogenic inducer, 61 variant), Prothomyosin alpha, α-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET and calsyntenin-1 in thyroid cancer patients' blood. Patient serum samples were depleted of the 20 most abundant blood proteins using the Proteoprep 20 Plasma Immunodepletion kit (Sigma-Aldrich, MO) according to manufacturer's specifications.

For Western Blot analysis, 12% SDS-PAGE gels were used as described earlier. Proteins were transferred from the gel to a polyvinylidenedifluoride (PVDF) membrane, that was blocked using 5% non-fat milk in Tris-buffered saline (TBS, 0.1 M, pH=7.2). Blots were incubated using monoclonal or polyclonal antibodies against E-cadherin, Nucleolin, CYR61 (cysteine rich angiogenic inducer, 61 variant), Prothomyosin alpha, α-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET and calsyntenin-1 at the appropriate dilutions at 4° C. for 2 hours. The membranes were incubated with horseradish peroxidase-conjugated anti-rabbit or anti-mouse secondary antibody (DAKO Cytomation, Denmark), diluted to an appropriate concentration in 1% BSA, and room-temperature incubated for 2 h. Following each step, blots were washed three times with Tween (0.1%)-Tris-buffer saline (TTBS). Protein bands were detected by the enhanced chemiluminescence method (ECL, Santa Cruz Biotechnology, CA) on XO-MAT film.

### RESULTS

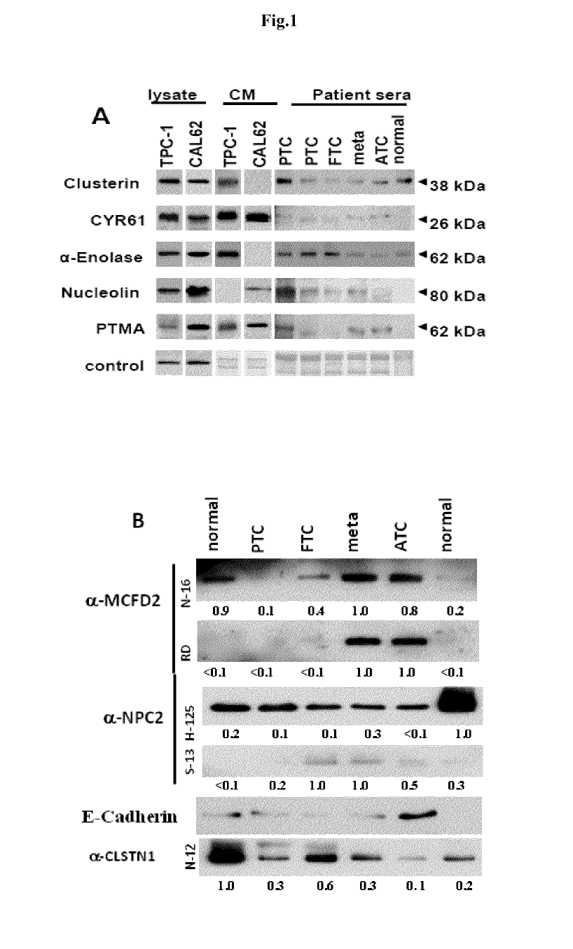

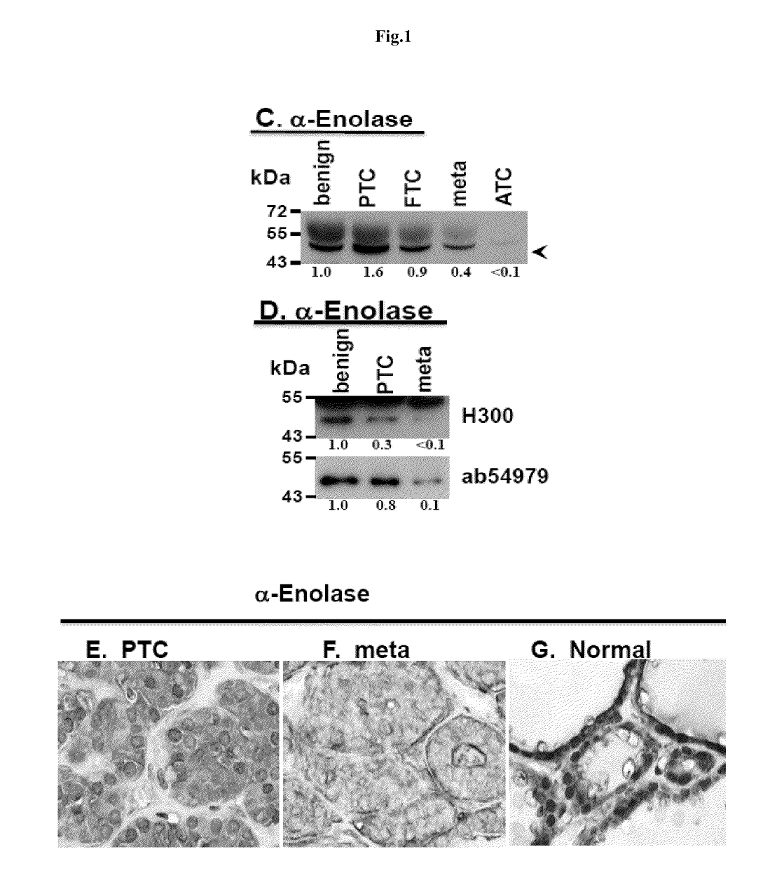

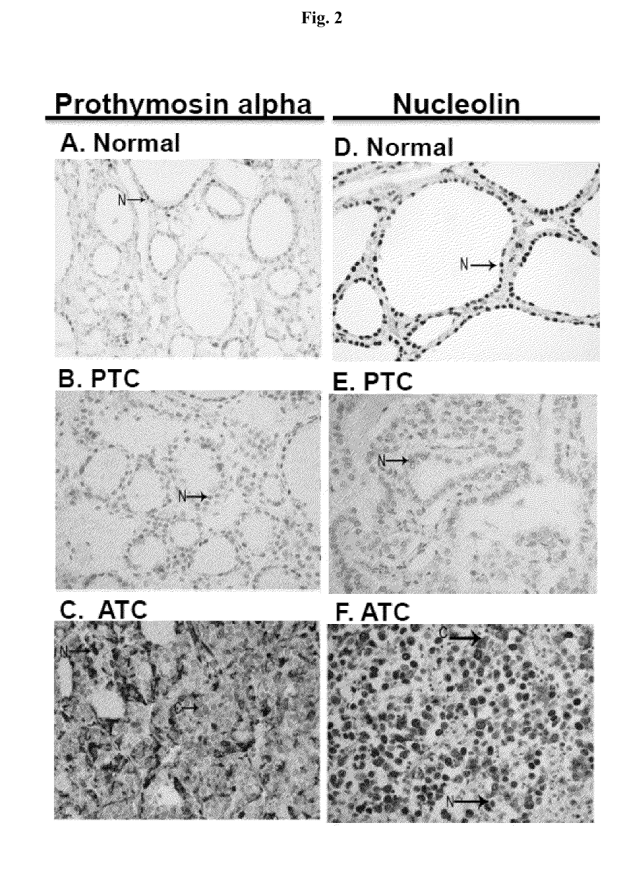

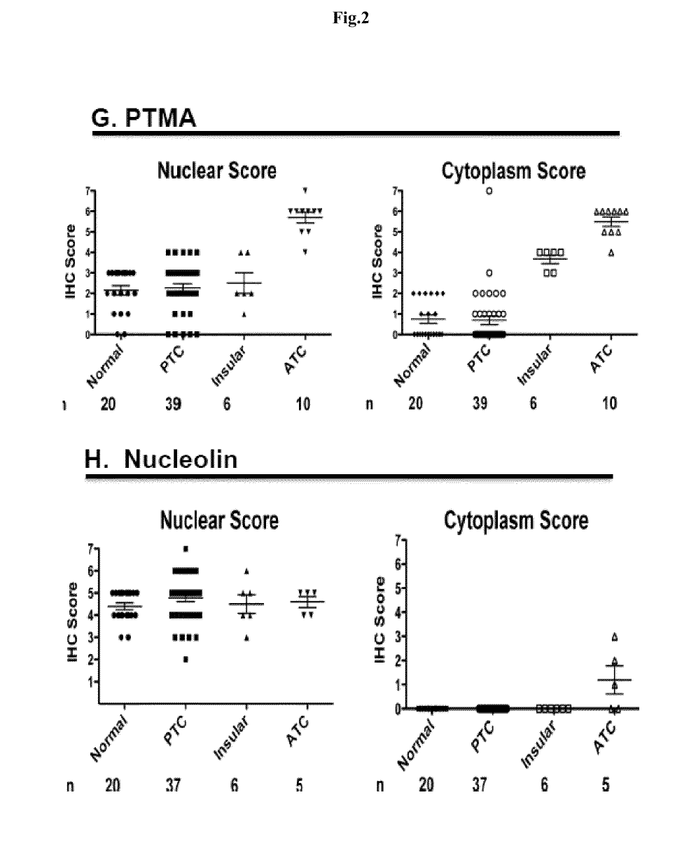

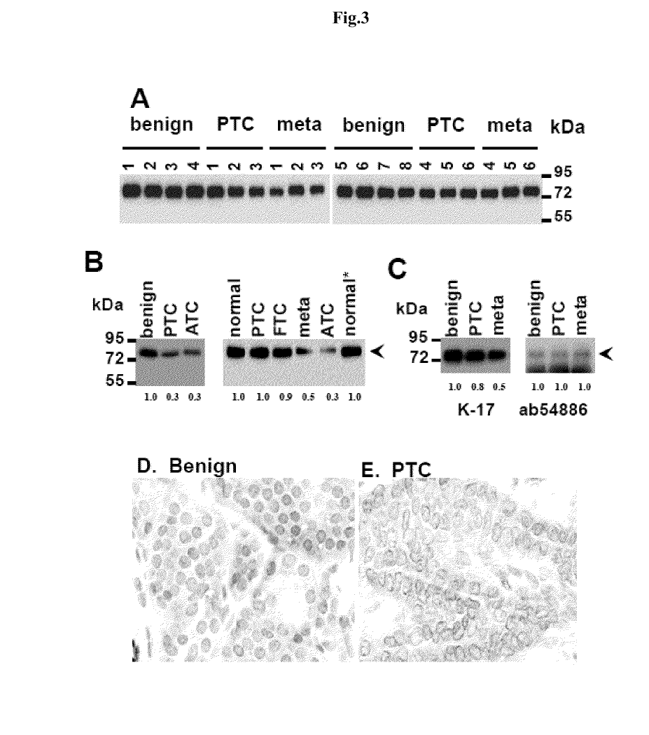

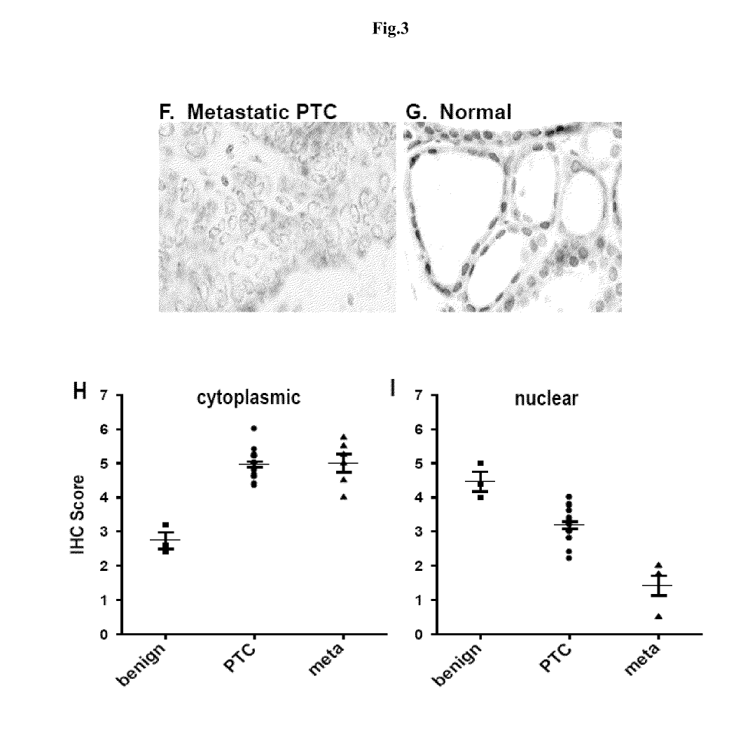

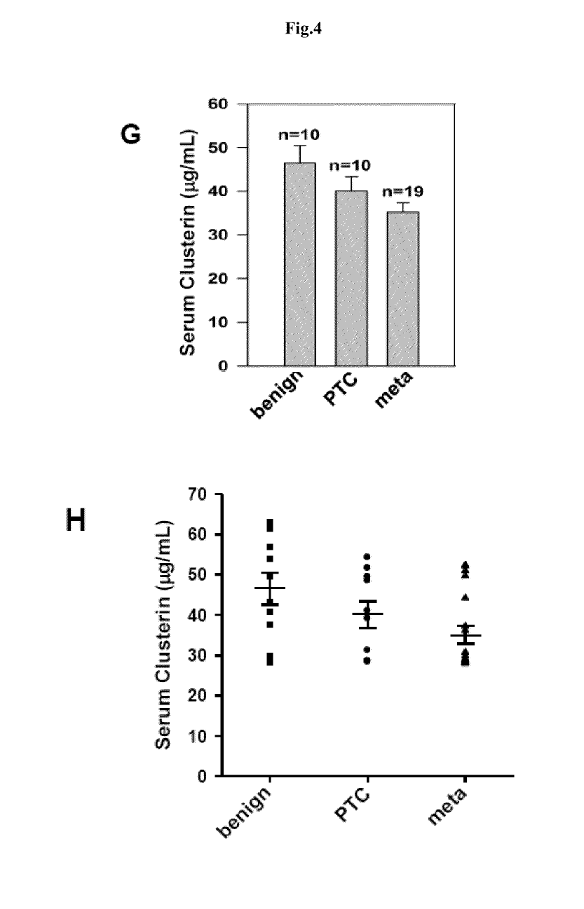

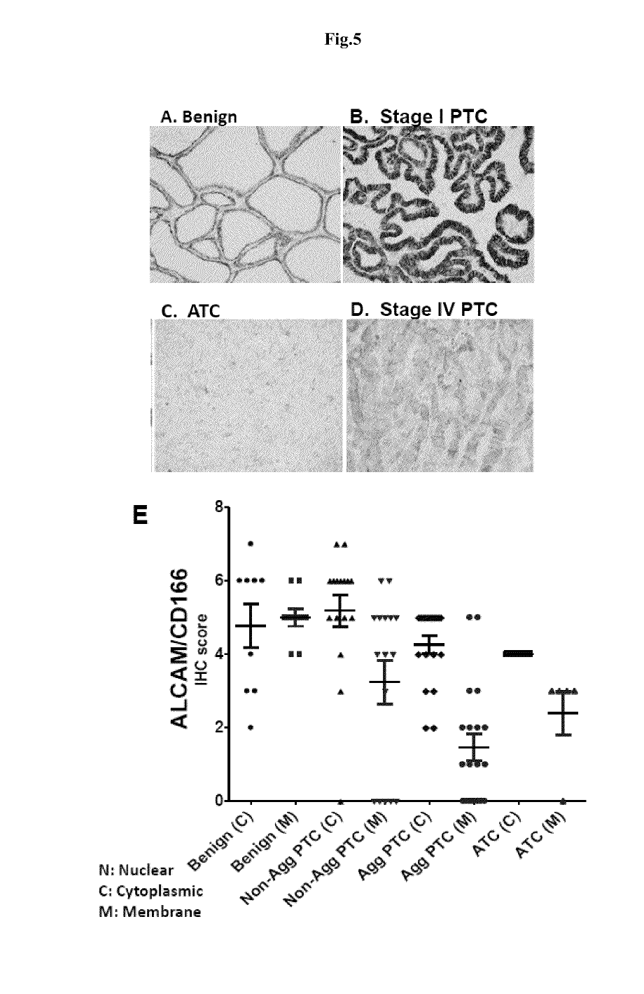

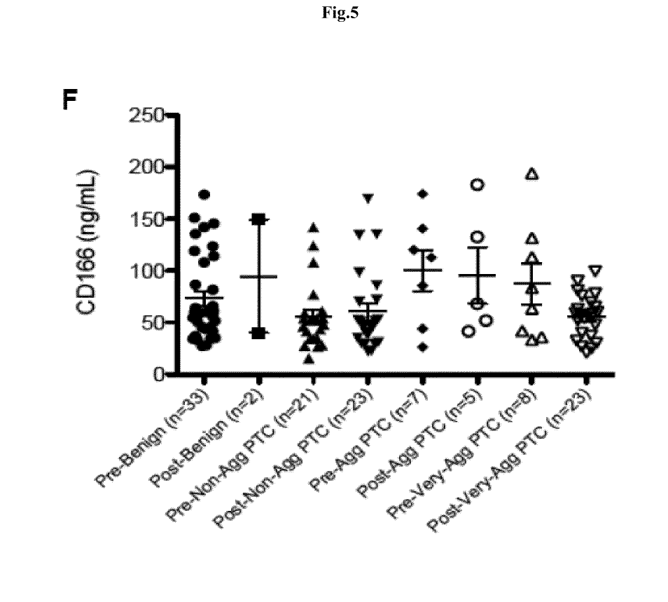

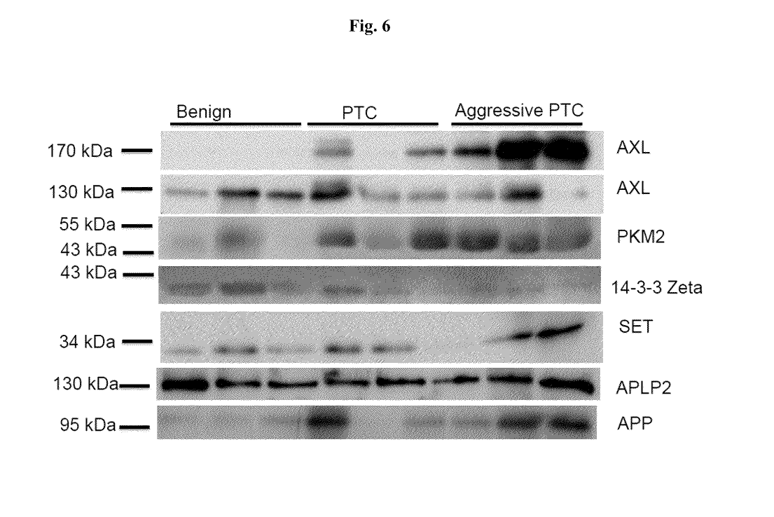

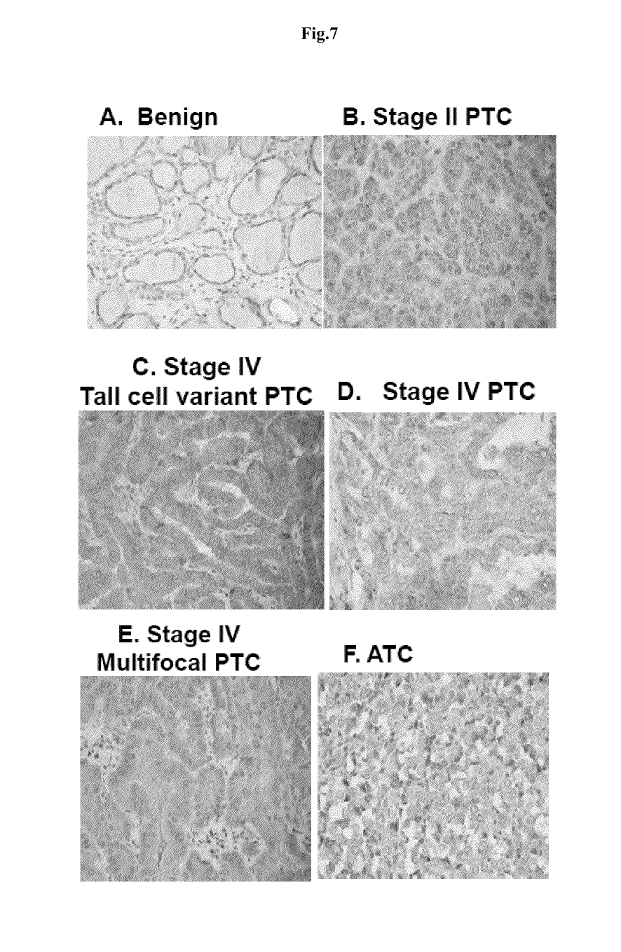

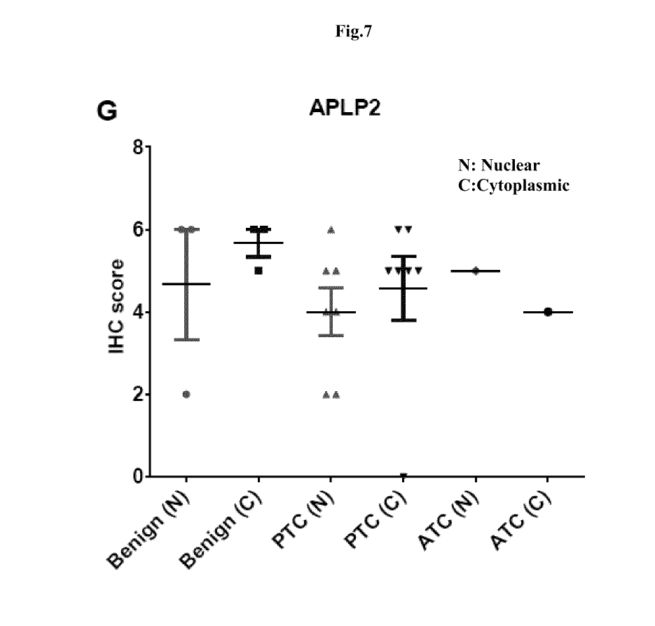

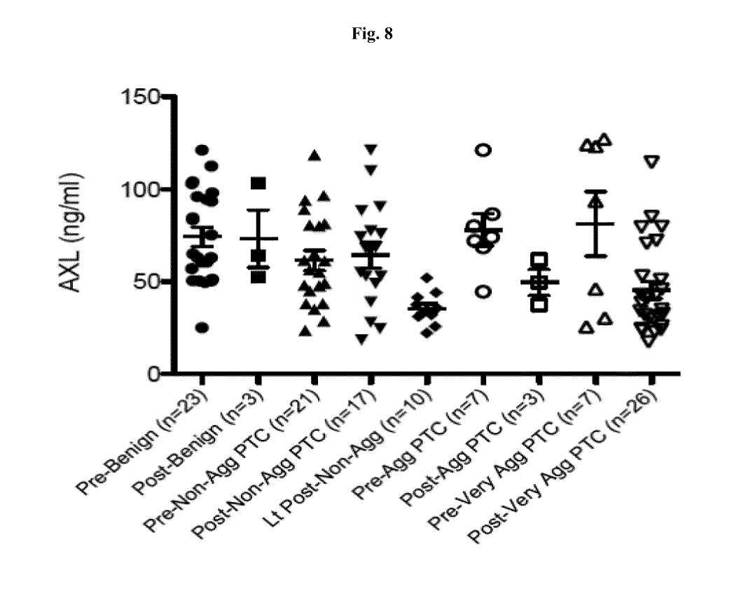

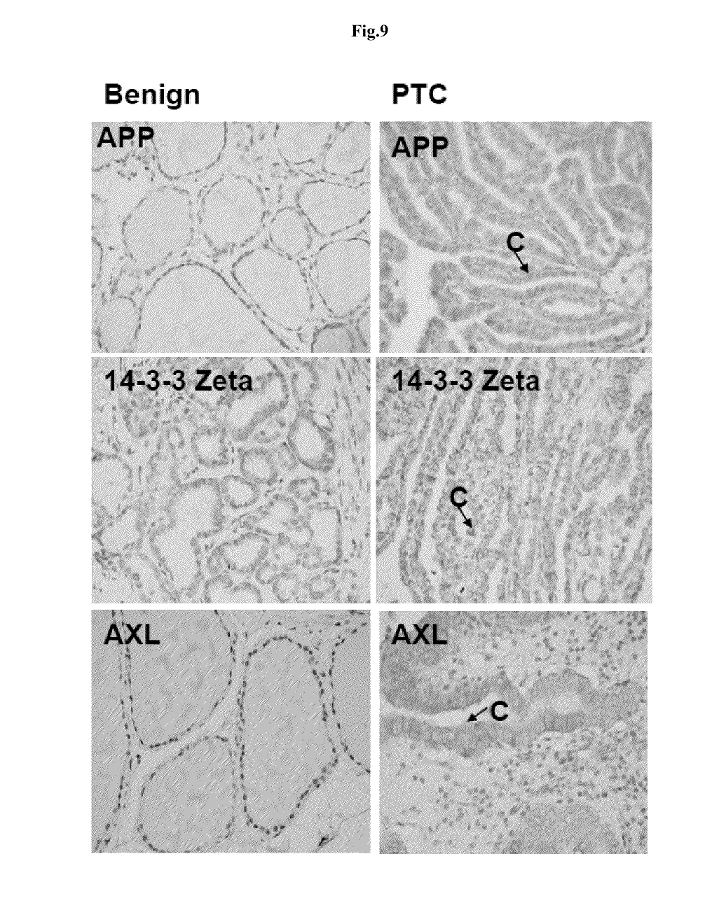

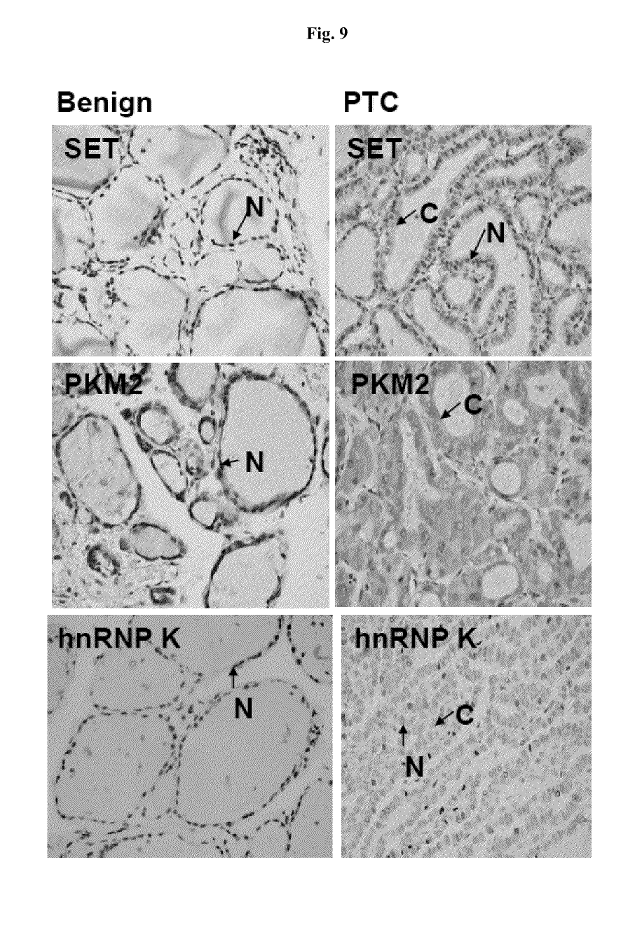

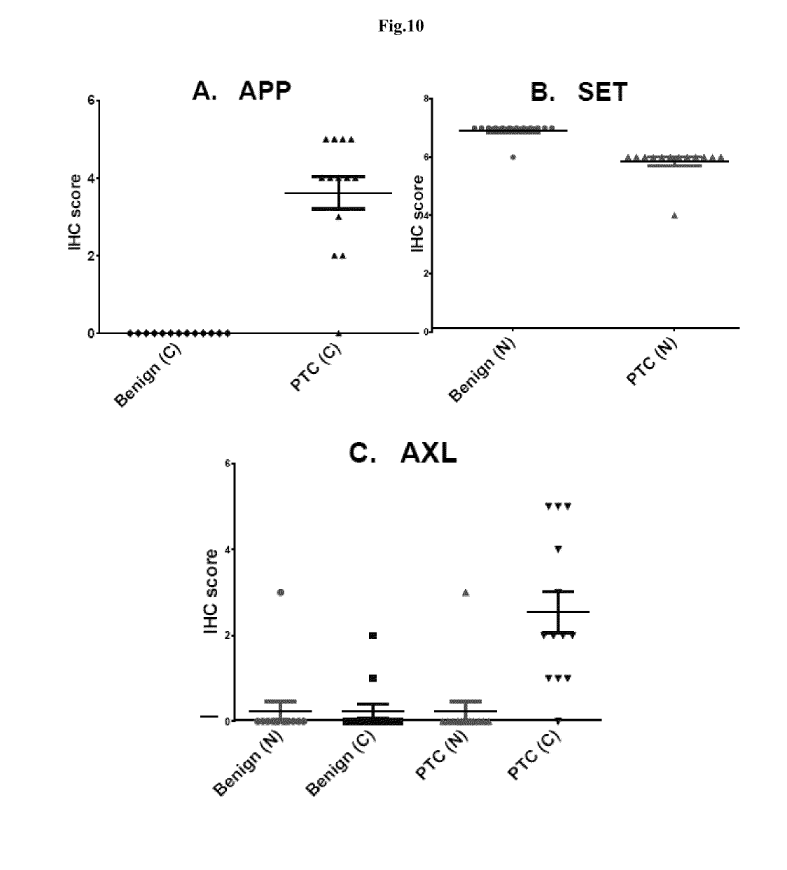

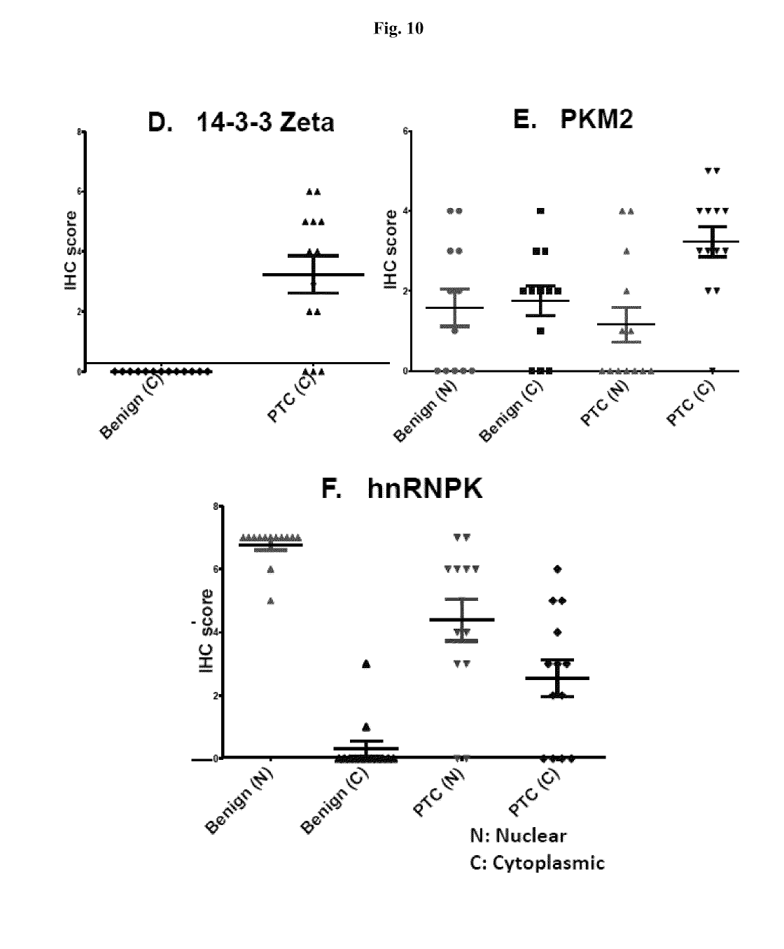

The results are discussed below and aspects are illustrated in FIGS. 1-11.

Optimization of Cell Culture Conditions for SFM Collection. While cells are normally grown in media that contains serum, the high abundance proteins found in serum would interfere with the detection of secretome proteins. For this reason, cell culture conditions needed to be optimized for SFM collection. To avoid this interference, the cells were washed thoroughly four times (three times with PBS and once with serum-free media) and then grown in serum-free media for 48 h, allowing secretome proteins to accumulate. To limit cellular stress under these conditions, cells were only placed in SFM when they reached 60% confluence. Trypan blue staining was performed following collection of the SFM at 48 h to estimate the number of dead cells. Since >95% of cells were viable at 48 h, the release of non-secretory proteins into the media is considered to be minimal, but cannot be completely ruled out.

Proteins Released by TPC-1, CAL 62, MRO, WRO, BCPAP, SW1736 and C643 Thyroid Cancer Cell Lines. A total of 233 proteins were initially identified in the four thyroid cancer cell lines. The subcellular localization and biological functions of the proteins were determined using Ingenuity Pathway Analysis (IPA, Ingenuity Systems, www.ingenuity.com). In all cell lines, membrane and extracellular proteins were predominantly identified. Additionally, proteins associated with cellular metabolism were common to all cell lines. Numerous signal transduction and cell cycle proteins were also identified in WRO and TPC-1 cells. In order to become a candidate for further verification, proteins must have been identified from MS spectrum data with at least 2 high-confidence peptides with a confidence level ≧95%. Proteins identified with at least two high-confidence peptides are considered high-confidence identifications. Proteins were not identified from the 0 h controls, except for blood albumin and globulins, which were removed from the identified proteins list. After applying the high confidence threshold to the identified protein list, 83 proteins remained as candidates for independent verification (see Table 1). Protein sequences were obtained for these proteins and inputted into SignalP and/or SecretomeP to obtain the reported score. Literature searches were performed on each protein to identify its cellular localization, and whether it has been reported to be present in exosomes or in patient blood/tissue samples. Nearly all of these high-confidence identifications were determined to be secretory proteins according to their SignalP and SecretomeP scores.

Verification of Selected Secretome Proteins in Human Sera by Western Blotting. The presence of select proteins were independently verified by Western Blot in thyroid cancer patients' sera, and in the SFM (see FIGS. 1A-D, 3A-C, 4A,B and 6). The proteins that have been verified are E-cadherin, Nucleolin, CYR61 (cysteine rich angiogenic inducer, 61 variant), Prothomyosin alpha, α-Enolase, Biotinidase, Clusterin, Tyrosine-protein kinase receptor UFO (AXL), amyloid precursor protein (APP), amyloid precursor protein like protein 2 (APLP2), Pyruvate kinase M2 (PKM2), α-MCFD2, α-NPC2, 14-3-3 zeta, SET, and calsyntenin-1. Proteins were selected for verification were based upon SecretomeP scores, SignalP scores, IPA database information, along with information from literature searches (i.e. whether the protein has also been reported to be present in the blood of thyroid cancer patients in other studies, whether it has been reported to be found in exosomes, and/or the role the protein may play in disease progression). The justification for selection of these proteins lies in the fact that they were high-confidence identifications, are suggested to be secreted proteins based upon their SignalP and/or SecretomeP scores, and/or have been reported in the literature to be secreted or to play a role in cancer pathogenesis.

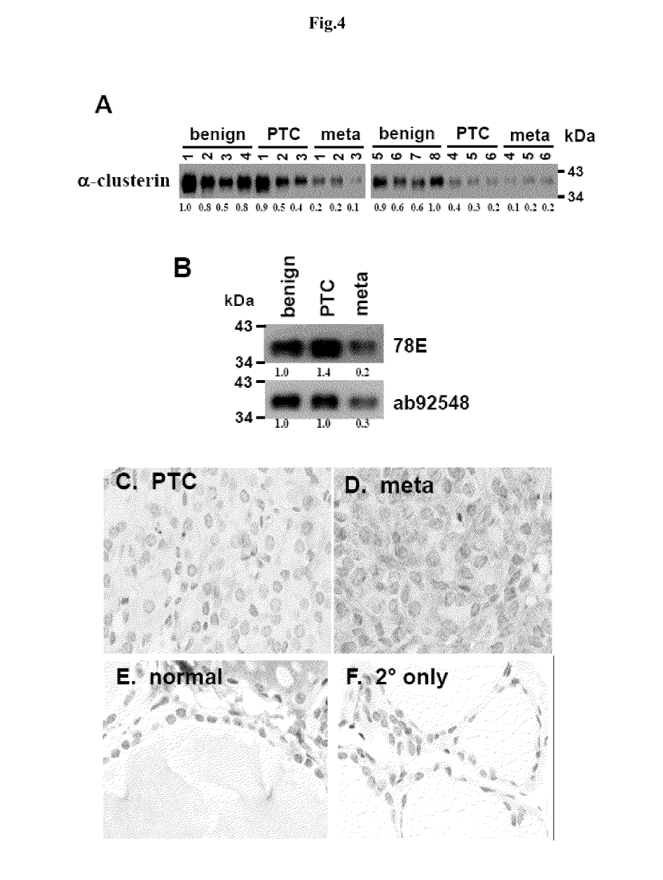

Verification of Selected Secretome Proteins in Human Sera by ELISA. The presence of select proteins were independently verified by ELISA in thyroid cancer patients' sera (see FIGS. 4G and H, 5F and 8). The proteins that have been verified are clusterin, ALCAM/CD166 and AXL tyrosine kinase.

Verification of Selected Secretome Proteins in Human Thyroid Carcinoma and Normal Tissues by Immunohistochemistry. The presence of select proteins were independently verified by immunohistochemistry in thyroid carcinoma, benign thyroid nodules and/or normal tissues (see FIGS. 1E-G, 2A-H, 3D-I, 4C-F, 5A-E, 7A-G, 9 and 10). The proteins that have been verified are α-Enolase, Prothomyosin alpha, Nucleolin, Biotinidase, Clusterin, ALCAM/CD166, amyloid precursor protein like protein 2 (APLP2), amyloid precursor protein (APP), 14-3-3 zeta, Tyrosine-protein kinase receptor UFO (AXL), SET, Pyruvate kinase M2 (PKM2), and Heterogeneous ribonucleoprotein K (hnRNP K).

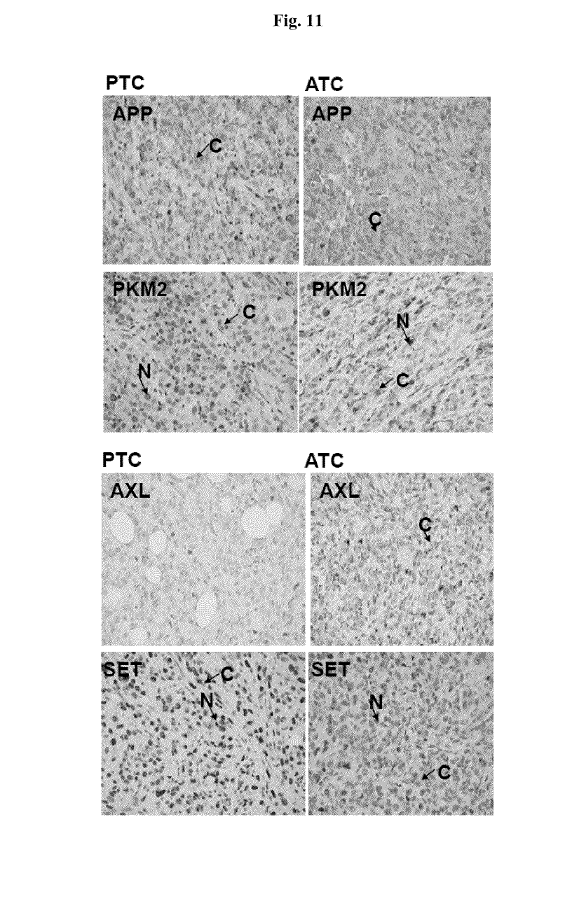

Verification of Selected Secretome Proteins in Mouse Xenografts of Human Thyroid Carcinoma Cell Lines. The presence of select proteins were independently verified by immunohistochemistry in tissue sections of xenografts of human thyroid carcinoma cell lines, BCPAP (papillary thyroid carcinoma) and C643 (anaplastic thyroid carcinoma) in immunocompromised mice (NOD/Scid/gamma) (see FIG. 11). The proteins that have been verified are amyloid precursor protein (APP), Tyrosine-protein kinase receptor UFO (AXL), Pyruvate kinase M2 (PKM2), and SET. The expression patterns and subcellular localization of these proteins in mouse xenografts were similar to those observed in cultured thyroid cancer cells and in human thyroid carcinomas confirming that these proteins retain their characteristics in xenografts.

### DISCUSSION

The study revealed a total of 233 proteins in the secretome of TPC-1, BCPAP, CAL 62, SW1736, WRO, and MRO cells, of which 83 are considered high-confidence identifications due to the numerous peptides used in their identification. Nearly all identified high-confidence proteins were deemed to be secretory according to their SignalP and SecretomeP scores, lending additional support to the hypothesis that the proteins identified in the study are secretory proteins. There were far more proteins unique to TPC-1 and SW1736 cells than the other cell lines due to the fact that three-times as many TPC-1 and SW1736 cells were used for SFM collection.

Nucleolin. Nucleolin is a nuclear protein involved in numerous cell cycle processes. It does not have a known classical secretion signal and is not suggested to be a secretory protein based upon its SecretomeP and SignalP scores. SecretomeP and SignalP scores cannot completely rule out the possibility a protein is in-fact secretory, and numerous studies have demonstrated that nucleolin is in-fact, present on the cellular surface of proliferating cells [6]. It remains unclear how nucleolin is transported from the nuclear membrane to the cell surface. It has been shown that the use of antagonists to surface nucleolin suppresses tumour growth and angiongenesis, suggesting an important role between cell-surface nucleolin expression and tumour progression [7]. Confirmatory western blotting revealed nucleolin to be present in all of the cell lysates, but only in the SFM of WRO and MRO cells. It was also detectable in all 5 thyroid cancer patient samples, but not in the normal blood sample. The detectablity of nucleolin in patient blood samples suggests it may be a useful thyroid cancer biomarker.

Cysteine Rich Angiogenic Inducer 61 (CYR61). CYR61 belongs to the CCN family of proteins, initially identified as secretory proteins whose production is induced by oncogenes [8]. Paradoxically, CYR61, while having demonstrated importance in cancer cell proliferation, has also been shown to play an important role in the induction of apotosis [9]. The secretome analysis revealed CYR61 to be secreted by TPC-1 cells. As with nucleolin, it was present in all thyroid cancer patient blood samples, but not in the normal. Interestingly, CYR61 was only found in the whole cell lysate of TPC-1 cells. This illustrates the potential for secretome analysis to reveal markers that may be used to distinguish between different thyroid cancer types.

E-Cadherin. The cadherins are a family of proteins responsible for cell-cell adhesion. Studies have shown that loss of E-cadherin mediated cell adhesion is associated with increased tumour aggressiveness and patient mortality [10]. E-Cadherin expression was noticed in the cell lysate and SFM of WRO and MRO cells, but not TPC-1. It was also present in all thyroid cancer blood samples, but not in the normal controls.

Prothymyosin alpha. Prothymyosin alpha is a heterochromatin remodelling protein whose expression has previously been shown to be significantly elevated in well-differentiated thyroid carcinomas compared to ademonas and goitres. [11] While it was present in the cell lysate of all three cell-lines, it was found to be secreted only in TPC-1 cells. As with nucleolin, CYR61, and E-Cadherin, prothymyosin alpha may serve as a potential thyroid cancer biomarker as it was found in all thyroid cancer patient blood samples but not in the normal controls.

Activated leukocyte cell adhesion molecule (ALCAM/CD166). Activated leukocyte cell adhesion molecule (ALCAM/CD166) is usually expressed in cells that are involved in growth and migration, including neural development, immune response, and tumor formation. [12, 13] It is an adhesion molecule that is located at intercellular junctions and is involved in tumor cell adhesion, which is necessary for primary tumor formation and metastasis. ALCAM binds to CD6 on T-cells and mediates T-cell activation and proliferation. ALCAM was identified in four thyroid cancer cell lines, including TPC-1, BCPAP, CAL62, and SW1736.

AXL. AXL is a receptor tyrosine kinase, ubiquitously expressed transmembrane protein, that binds to growth factors and transduces signals from the extracellular matrix to the cytoplasm. It is involved in stimulating cell proliferation and aggregation through hemophilic binding. AXL overexpression plays a role in cell adhesion and overexpression of this protein has been found in several cancers. [14]

APP. Amyloid beta (A4) protein—is a cell surface receptor and transmembrane precursor protein that is cleaved by different secretases to form a variety of peptides which can bind to complexes for transcriptional activation. APP plays a role in development of the adult nervous system, cell adhesion, neuronal survival, neurite outgrowth, synaptogenesis, vesicular transport, neuronal migration, modulation of synaptic plasticity, and insulin and glucose homeostasis. [15]

PKM2. Normal cells express the pyruvate kinase M1 isoform (PKM1), tumor cells predominantly express the M2 isoform (PKM2). Switching from PKM1 to PKM2 promotes aerobic glycolysis and provides a selective advantage for tumor formation. The PKM1/M2 isoforms are generated through alternative splicing of two mutually exclusive exons. A recent study shows that the alternative splicing event is controlled by heterogeneous nuclear ribonucleoprotein (hnRNP) family members hnRNPA1, hnRNPA2, and polypyrimidine tract binding protein (PTB; also known as hnRNPI). [16]

APLP2. Amyloid-like protein 2 (APLP2) is a paralogue of APP and is similarly cleaved by secretases to form peptides which may have similar functions to APP cleaved domains, including cell adhesion, migration, cell signaling, and cell cycle regulation. Increased expression of APLP2 has been reported in some tumours. APLP2 was identified in seven cell lines, including TPC-1, BCPAP, CAL62, SW1736, C643, MRO, and WRO. [17]

Clusterin. Clusterin is a glycoprotein that has many biological functions of which are not well understood. It appears to be involved in cell death, tumour progression, tissue differentiation, cell-cell interactions, cell proliferation, lipid transportation, and neurodegenerative disorders. Clusterin was identified in seven cell lines, including TPC-1, CAL62, SW1736, MRO, and WRO. [18]

In summary, by verifying the above protein biomarkers in the sera and tissues of thyroid cancer patients, the feasability of using a secretome approach to identify potential thyroid cancer biomarkers has been illustrated. The findings also reveal the potential for secretome analysis to identify proteins that may help to distinguish between aggressive and non-aggressive carcinomas.

TABLE 1
Novel Thyroid Cancer Markers

| ID | SEQ ID No. | Thyroid Cancer Marker | Accession No. |
|---|---|---|---|
| 1. | SEQ ID-31 | Versican | trm\|Q59FG9 |
| 2. | SEQ ID-6 | Clusterin | spt\|P10909 |
| 3. | SEQ ID-91 | Vacuolar proton pump subunit S1 | spt\|Q15904 |
| 4. | SEQ ID-32 | Gamma-glutamyl hydrolase* | spt\|Q92820 |
| 5. | SEQ ID-66 | Insulin-like growth factor binding | spt\|Q16270 |
| | | protein 7 | |
| 6. | SEQ ID-21 | α-Enolase*** | trm\|Q53FT9 |
| 7. | SEQ ID-82 | Stem cell growth Factor | trm\|Q5U0B9 |
| 8. | SEQ ID-34 | Syndecan-4 | trm\|Q53FN9\|Q53FN9_HUMAN; |
| | | | spt\|P31431\|SDC4_HUMAN |
| 9. | SEQ ID-28 | Fibronectin | trm\|Q6N025 |
| 10. | SEQ ID-70 | Nucleophosmin | spt\|P06748 |
| 11. | SEQ ID-29 | Ubiquitin-A 52-residue ribosomal | trm\|Q3MIH3 |
| | | protein fusion product | |
| 12. | SEQ ID-33 | Lysyl oxidase-like 2* | trm\|Q53HV3\|Q53HV3_HUMAN; |
| | | | spt\|Q9Y4K0\|LOXL2_HUMAN |
| 13. | SEQ ID-18 | Nucleobindin-1 variant | trm\|Q53GX6 |
| 14. | SEQ ID-14 | Calsyntenin-1 | trm\|Q5UE58 |
| 15. | SEQ ID-3 | Prothymosin-α | trm\|Q9NYD3 |
| 16. | SEQ ID-40 | Agrin | spt\|O00468 |
| 17. | SEQ ID-7 | Amyloid-like protein 2 | trm\|Q9BT36 |
| | | (APLP2)*** | |
| 18. | SEQ ID-43 | Beta-2-microglobulin | trm\|Q6IAT8\|Q6IAT8_HUMAN; |
| | | | spt\|P61769\|B2MG_HUMAN |
| 19. | SEQ ID-27 | CD44 antigen | spt\|P16070 |
| 20. | SEQ ID-22 | dystroglycan 1 | trm\|Q969J9 |
| 21. | SEQ ID-17 | Gelsolin** | spt\|P06396 |
| 22. | SEQ ID-35 | hnRNP A2/B1** | spt\|P22626 |
| 23. | SEQ ID-8 | Nucleolin | spt\|P19338 |
| 24. | SEQ ID-11 | SET protein | trm\|Q6FHZ5 |
| 25. | SEQ ID-5 | Biotinidase* | spt\|P43251 |
| 26. | SEQ ID-16 | Nidogen-1* | spt\|P14543 |
| 27. | SEQ ID-15 | Dickkopf-related protein 3 | trm\|Q6PQ81 |
| 28. | SEQ ID-30 | Basement membrane specific | spt\|P98160 |
| | | heparin sulfate core protein** | |
| 29. | SEQ ID-45 | Cadherin 2*** | spt\|P19022 |
| 30. | SEQ ID-62 | Granulins (proepithelin)*** | spt\|P28799 |
| 31. | SEQ ID-2 | Activated Leukocyte Cell | trm\|Q1HGM9 |
| | | Adhesion Molecule (ALCAM) | |
| 32. | SEQ ID-48 | Cathepsin Z*** | trm\|Q5U000 |
| 33. | SEQ ID-64 | Hypothetical protein (belongs to | trm\|Q8WVW5 |
| | | the actin family)*** | |
| 34. | SEQ ID-73 | Peptidylproylisomerase A | trm\|Q3KQW3 |
| 35. | SEQ ID-65 | Insulin-like growth factor binding | spt\|P24592 |
| | | protein 6*** | |
| 36. | SEQ ID-1 | AXL receptor tyrosine kinase*** | trm\|Q8N5L2\|Q8N5L2_HUMAN |
| 37. | SEQ ID-38 | 14-3-3 protein epsilon (14-3-3E) | spt\|P62258 |
| 38. | SEQ ID-10 | 14-3-3 protein zeta/delta (Protein | spt\|P63104 |
| | | kinase C inhibitor protein 1)*** | |
| 39. | SEQ ID-39 | 60S acidic ribosomal protein P2 | spt\|P05387 |
| | | (NY-REN-44 antigen)*** | |
| 40. | SEQ ID-41 | Alpha-actinin-1*** | spt\|P12814 |
| 41. | SEQ ID-42 | Alpha-actinin-4 | spt\|O43707 |
| 42. | SEQ ID-44 | C4B1 (Complement component | trm\|Q6U2E9 |
| | | C4B)*** | |
| 43. | SEQ ID-26 | Calmodulin (CaM)*** | trm\|Q9BRL5\|Q9BRL5_HUMAN; |
| | | | spt\|P62158\|CALM_HUMAN |
| 44. | SEQ ID-46 | Calreticulin (CRP55) | trm\|Q53G71 |
| 45. | SEQ ID-47 | Cathepsin C*** | trm\|Q8WY99 |
| 46. | SEQ ID-49 | CDNA FLJ45706 fis, clone | trm\|Q6ZS99 |
| | | FEBRA2028457, highly similar to | |
| | | Nucleolin*** | |
| 47. | SEQ ID-50 | Chaperonin 10-related protein*** | trm\|Q9UNM1 |
| 48. | SEQ ID-51 | Cofilin-129 | spt\|P23528 |
| 49. | SEQ ID-52 | Collagen alpha-1 (V) chain*** | spt\|P20908 |
| 51. | SEQ ID-54 | Collagen alpha-1(XII) chain*** | spt\|Q99715 |
| 52. | SEQ ID-55 | Collagen, type I, alpha 2*** | trm\|Q7Z5S6 |
| 53. | SEQ ID-56 | Colony stimulating factor 1 | trm\|Q5VVF4 |
| | | (Macrophage)*** | |
| 54. | SEQ ID-57 | EGF-containing fibulin-like | spt\|Q12805 |
| | | extracellular matrix protein 1*** | |
| 55. | SEQ ID-58 | Filamin A*** | trm\|Q60FE6 |
| 56. | SEQ ID-59 | Follistatin-related protein 1*** | spt\|Q12841 |
| 57. | SEQ ID-60 | Fructose-bisphosphate aldolase*** | trm\|Q6FI10 |
| 58. | SEQ ID-63 | Heat shock protein (HSP 90-alpha | trm\|Q5CAQ7 |
| | | 2)*** | |
| 59. | SEQ ID-61 | Glucose-6-phosphate isomerase | spt\|P06744 |
| 60. | SEQ ID-13 | HNRPK protein (Heterogeneous | trm\|Q5T6W2 |
| | | nuclear ribonucleoprotein K)*** | |
| 61. | SEQ ID-80 | Secretogranin 2 | trm\|Q53T11 |
| 62. | SEQ ID-67 | L-lactate dehydrogenase A chain | spt\|P00338 |
| 63. | SEQ ID-68 | Matrix metalloproteinase 1 | trm\|Q5TZP0 |
| | | (MMP-1) | |
| 64. | SEQ ID-69 | Matrix metalloproteinase 1 | trm\|Q53G75 |
| | | preprotein variant | |
| 65. | SEQ ID-37 | Niemann-Pick disease, type C2 | trm\|Q53HV6 |
| | | variant*** | |
| 66. | SEQ ID-71 | Nucleoside diphosphate kinase | trm\|Q32Q12 |
| | | (NME1-NME2)*** | |
| 67. | SEQ ID-72 | OAF homolog*** | trm\|Q86UD1 |
| 68. | SEQ ID-74 | Phosphoglycerate kinase*** | trm\|Q5J7W1 |
| 69. | SEQ ID-75 | PKM2 protein*** | trm\|Q8WUW7 |
| 70. | SEQ ID-85 | Tissue-type plasminogen activator | trm\|Q6PJA5 |
| | | (PLAT protein)*** | |
| 71. | SEQ ID-76 | Protein CutA*** | spt\|O60888 |
| 72. | SEQ ID-77 | Protein FAM3C*** | spt\|Q92520 |
| 73. | SEQ ID-78 | Protein S100-A9*** | spt\|P06702 |
| 74. | SEQ ID-12 | Pyruvate kinase isozymes | trm\|Q53GK4 |
| | | M1/M2*** | |
| 75. | SEQ ID-79 | Ribosomal protein S27a*** | spt\|P62979 |
| 76. | SEQ ID-81 | SPARC (Secreted protein acidic | spt\|P09486 |
| | | and rich in cysteine) (Osteonectin) | |
| 77. | SEQ ID-83 | Sulfhydryl oxidase 1 (Quiescin | spt\|O00391 |
| | | Q6) (hQSOX)*** | |
| 78. | SEQ ID-84 | Thrombospondin 2*** | trm\|Q5RI52 |
| 79. | SEQ ID-86 | Transforming growth factor, beta- | trm\|Q53GU8 |
| | | induced, 68 kDa variant | |
| 80. | SEQ ID-87 | Transketolase (TK)*** | trm\|Q53EM5 |
| 81. | SEQ ID-88 | Translation elongation factor 1 | trm\|Q96RE1 |
| | | alpha 1-like 14*** | |
| 82. | SEQ ID-89 | Triosephosphate isomerase | trm\|Q6FHP9 |
| 83. | SEQ ID-90 | UV excision repair protein | spt\|P54727 |
| | | RAD23 homolog B*** | |
| 84. | SEQ ID-19 | Cysteine rich angiogenic inducer, | trm\|Q53FA4\|Q53FA4_HUMAN; |
| | | 61 variant (CYR61) | spt\|O00622\|CYR61_HUMAN |
| 85. | SEQ ID-23 | Melanoma associated antigen | rm\|Q92626\|Q92626_HUMAN |
| 86. | SEQ ID-24 | Osteopontin | trm\|Q0JV14\|Q0JV14_HUMAN; |
| | | | spt\|P10451\|OSTP_HUMAN |
| 87. | SEQ ID-25 | Plasminogen activator, urokinase | trm\|Q5SWW9\|Q5SWW9_HUMAN; |
| | | activator | trm\|Q5SWW8\|Q5SWW8_HUMAN; |
| | | | trm\|Q5PY49\|Q5PY49_HUMAN; |
| | | | trm\|Q53XS3\|Q53XS3_HUMAN; |
| | | | spt\|P00749\|UROK_HUMAN |
| 88. | SEQ ID-36 | MCFD2*** | trm\|Q68D61 |

*Novel Papillary Cancer Markers (TPC-1/BCPAP);
**Novel Follicular Cancer Markers (MRO/WRO);
***Novel Aggressive/Metastatic Thyroid Cancer Markers (CAL62, SW1736, C643)

TABLE 2
Known Thyroid Cancer Markers

| S. No | SEQ ID# | Protein | Protein ID |
|---|---|---|---|
| 1. | SEQ ID 4 | Galectin-3 | spt\|Q08380 |
| 2. | SEQ ID 92 | Serum thyroglobulin | spt\|p01266 |
| 3. | SEQ ID 93 | BRAF mutation | spt\|p15056 |
| 4. | SEQ ID 20 | E-Cadherin | spt\|P12830 |
| 5. | SEQ ID 94 | Vimentin | trm\|Q5JVT0 |
| 6. | SEQ ID 95 | Galectin-1 | spt\|p09382 |
| 7. | SEQ ID 9 | Amyloid precursor | AAB19991 |
| | | protein (APP) | |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

### CITATIONS FOR PUBLICATIONS REFERRED TO IN THE SPECIFICATION

1. Damante, G. S., A.; Tell, G.; Thyroid Tumors: novel insights from proteomics studies. Expert Rev Proteomics 2009, 6 (4), 363-376.
2. Carpi, A.; Di Coscio, G.; Iervasi, G.; Antonelli, A.; Mechanick, J.; Sciacchitano, S.; Nicolini, A., Thyroid fine needle aspiration: how to improve clinicians' confidence and performance with the technique. Cancer Lett 2008, 264 (2), 163-71.
3. Nikiforova, M. N.; Nikiforov, Y. E., Molecular diagnostics and predictors in thyroid cancer. Thyroid 2009, 19 (12), 1351-61.
4. Li, H. D., L. V.; Ghanny, S.; Li, W.; Romaschin, A. D.; Colgan, T. J.; Siu, K. W., Identification of candidate biomarker proteins released by human endometrial and cervical cancer cells using two-dimensional liquid chromatography/tandem mass spectrometry. J. Proteome Res. 2007, 7, 2615-2622.
5. (a) DeSouza, L.; Diehl, G.; Rodrigues, M. J.; Guo, J.; Romaschin, A. D.; Colgan, T. J.; Siu, K. W., Search for cancer markers from endometrial tissues using differentially labeled tags iTRAQ and cICAT with multidimensional liquid chromatography and tandem mass spectrometry. J Proteome Res 2005, 4 (2), 377-86; (b) DeSouza, L. V.; Grigull, J.; Ghanny, S.; Dube, V.; Romaschin, A. D.; Colgan, T. J.; Siu, K. W., Endometrial carcinoma biomarker discovery and verification using differentially tagged clinical samples with multidimensional liquid chromatography and tandem mass spectrometry. Mol Cell Proteomics 2007, 6 (7), 1170-82.
6. (a) Soundararajan, S.; Wang, L.; Sridharan, V.; Chen, W.; Courtenay-Luck, N.; Jones, D.; Spicer, E. K.; Fernandes, D. J., Plasma membrane nucleolin is a receptor for the anticancer aptamer AS1411 in MV4-11 leukemia cells. Mol Pharmacol 2009, 76 (5), 984-91; (b) Fogal, V.; Sugahara, K. N.; Ruoslahti, E.; Christian, S., Cell surface nucleolin antagonist causes endothelial cell apoptosis and normalization of tumor vasculature. Angiogenesis 2009, 12 (1), 91-100.
7. Destouches, D.; El Khoury, D.; Hamma-Kourbali, Y.; Krust, B.; Albanese, P.; Katsoris, P.; Guichard, G.; Briand, J. P.; Courty, J.; Hovanessian, A. G., Suppression of tumor growth and angiogenesis by a specific antagonist of the cell-surface expressed nucleolin. PLoS One 2008, 3 (6), e2518.
8. Chen, C.-C.; Lau, L. F., Functions and mechanisms of action of CCN matricellular proteins. The International Journal of Biochemistry & Cell Biology 2009, 41 (4), 771-783.
9. Leask, A., A sticky situation: CCN1 promotes both proliferation and apoptosis of cancer cells. Journal of Cell Communication and Signaling.
10. Makrilia, N.; Kollias, A.; Manolopoulos, L.; Syrigos, K., Cell adhesion molecules: role and clinical significance in cancer. Cancer Invest 2009, 27 (10), 1023-37.
11. Letsas, K. P.; Vartholomatos, G.; Tsepi, C.; Tsatsoulis, A.; Frangou-Lazaridis, M., Fine-needle aspiration biopsy-RT-PCR expression analysis of prothymosin alpha and parathymosin in thyroid: novel proliferation markers? Neoplasma 2007, 54 (1), 57-62.
12. Ofori-Acquah S F, King J A. Activated leukocyte cell adhesion molecule: a new paradox in cancer. Transl Res. 2008 March; 151(3):122-8.
13. Swart G W. Activated leukocyte cell adhesion molecule (CD166/ALCAM): Developmental and mechanistic aspects of cell clustering and cell migration. European Journal of Cell Biology 81,313±321.
14. Hafizi S, Dahlbäck B. Signalling and functional diversity within the Axl subfamily of receptor tyrosine kinases. Cytokine & Growth Factor Reviews 17 (2006) 295-304.
15. Keun-A Chang & Yoo-Hun Suh, Possible roles of amyloid intracellular domain of amyloid. BMB Rep. 2010 October; 43(10):656-63.
16. Chen M, Zhang J, Manley J L. Turning on a fuel switch of cancer hnRNP proteins regulate alternative splicing of pyruvate kinase mRNA. Cancer Res. 2010 Nov. 15; 70(22):8977-80.
17. Amit Tuli•Mahak Sharma•Xiaojian Wang•Laura C. Simone•Haley L. Capek Steven Cate•William H. Hildebrand•Naava Naslaysky•Steve Caplan•Joyce C. Solheim, Amyloid precursor-like protein 2 association with HLA class 1 molecules, Cancer Immunol Immunother (2009) 58:1419-1431.
18. Federica Rizzi and Saverio Bettuzzi, The clusterin paradigm in prostate and breast carcinogenesis, Endocr Relat Cancer. 2010 Jan. 29; 17 (1):R1-17.

What is claimed is:

1. A method for diagnosing thyroid cancer in a subject comprising:
detecting in a sample from the subject one or more Thyroid Cancer Markers as set out in Table 1; and
comparing the detected amount with an amount detected for a predetermined standard.

2. A method of claim 1, wherein in step (a) the Thyroid Cancer Markers are detected by contacting the sample with a reagent capable of measuring levels of the Thyroid Cancer Markers; and wherein a diagnosis of thyroid cancer is determined based on an increase in the level of at least one of the Thyroid Cancer Markers in the sample from the subject over the predetermined standard.

3. A method of claim 1 wherein the Thyroid Cancer Marker is a polypeptide detected by the following steps:
contacting the sample with an antibody that specifically binds to the polypeptide or a part thereof; and
detecting in the sample an amount of polypeptide that binds to the antibody, relative to a predetermined standard, and therefrom diagnosing thyroid cancer in the subject.

4. A method of claim 1 wherein the predetermined standard comprises levels detected in a subject without thyroid cancer, a subject with a lower grade of thyroid cancer or the same subject at a different time.

5. A method of claim 1 wherein the thyroid cancer is aggressive or metastatic thyroid cancer and the Thyroid Cancer Markers are chosen from activated leukocyte cell adhesion molecule (ALCAM)/CD166, tyrosine-protein kinase receptor UFO (AXL), amyloid precursor protein like protein 2 (APLP2), amyloid precursor protein (APP), biotinidase, cadherin-2, prothymosin-alpha, clusterin, syndecan-4, E-cadherin, gelsolin, hnRNP A2/B1, nucleolin, pyruvate kinase M2, α-enolase, 14-3-3 zeta, α-MCFD2, α-NPC2, calsyntenin and SET protein.

6. A method as claimed in claim 1 wherein the thyroid cancer is papillary thyroid cancer and the Thyroid Cancer Markers are chosen from gamma-glutamyl hydrolase, lysyl oxidase-like 2, biotinidase and nidogen-1, and optionally CYR61 and/or E-cadherin.

7. A method as claimed in claim 1 wherein the thyroid cancer is follicular thyroid cancer and the Thyroid Cancer Markers are chosen from gelsolin, hnRNP A2/B1, and basement membrane specific heparin sulfate core protein.

8. A method of claim 1 wherein the sample is a biological fluid.

9. A method of claim 8 wherein the sample is serum.

10. A method for detecting or characterizing a thyroid cancer in a patient comprising determining the status of Thyroid Cancer Markers in a sample obtained from the patient, wherein an abnormal status in the sample indicates the presence of the condition.

11. A kit for detecting and/or diagnosing thyroid cancer comprising agents that hybridize to or amplify polynucleotides encoding Thyroid Cancer Markers as set out in Table 1.

12. A method for detecting thyroid cancer in a subject comprising:
contacting the sample with antibodies that bind to Thyroid Cancer Markers as set out in Table 1, or polynucleotides that hybridize to or amplify polynucleotides Thyroid Cancer Markers as set out in Table 1; and
detecting in a sample from the subject one or more Thyroid Cancer Markers as set out in Table 1 wherein the antibody binding or amplification of the polynucleotides is indicative of the presence of a Thyroid Cancer Marker.

13. A method of diagnosing Thyroid Cancer in the subject of claim 12 wherein the presence of a Thyroid Cancer Marker in the sample as compared to a predetermined control is indicative of Thyroid Cancer in the subject.


SEQUENCE LISTING (95 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRRGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 3 - protein, 105 aa, Homo sapiens
MSDAAVDTSSEITTEDLKEKKEVVEEAENGRDAPAHGNANEENGEPEADNEVDEEEEEGGEEEGDGEEEDGDEDEGAESATGKRAAEDDEDDDVDTQKQKTDEDD

SEQ ID NO: 4 - protein, 151 aa, Homo sapiens
MRFLAATFLLLALSTAAQAEPVQFRDCGSVDGVIKEVNVSPCPTQPCQLSKGQSYSVNVTFTSNVQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 5 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 6 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 7 - protein, 522 aa, Homo sapiens [sequence omitted]

SEQ ID NO: 8 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 9 - protein, 49 aa, Homo sapiens
VFFAEDVGSNKGAIIGLMVGGVVIATVIFITLVMLKKKQYTSIHHGVVE

SEQ ID NO: 10 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 11 - protein, 277 aa, Homo sapiens
MSAQAAKVSKKELNSNHDGADETSEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 12 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKRKGADFLVTEVENGGSLGSKKGVNLPGAAVDLPAVSEKDIQDLKFGVEQDVDMVFASFIRKASDVREVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 13 - protein, 379 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSII

SEQ ID NO: 14 - protein, 971 aa, Homo sapiens
MLRRPAPALAPAARLLLAGLLCGGGVWAARVNKHKPWLEPTYHGIVTENDNTVLLDPPLIALDKDAPLRFAGEICGFKIHGQNVPFDAVVVDKSTGEGVIRSKEKLDCELQKDYSFTIQAYDCGKGPDGTNVKKSHKATVHIQVNDVNEYAPVFKEKSYKATVIEGKQYDSILRVEAVDADCSPQFSQICSYEIITPDVPFTVDKDGYIKNTEKLNYGKEHQYKLTVTAYDCGKKRATEDVLVKISIKPTCTPGWQGWNNRIEYEPGTGALAVFPNIHLETCDEPVASVQATVELETSHIGKGCDRDTYSEKSLHRLCGAAAGTAELLPSPSGSLNWTMGLPTDNGHDSDQVFEFNGTQAVRIPDGVVSVSPKEPFTISVWMRHGPFGRKKETILCSSDKTDMNRHHYSLYVHGCRLIFLFRQDPSEEKKYRPAEFHWKLNQVCDEEWHHYVLNVEFPSVTLYVDGTSHEPFSVTEDYPLHPSKIETQLVVGACWQEFSGVENDNETEPVTVASAGGDLHMTQFFRGNLAGLTLRSGKLADKKVIDCLYTCKEGLDLQVLEDSGRGVQIQAHPSQLVLTLEGEDLGELDKAMQHISYLNSRQFPTPGIRRLKITSTIKCFNEATCISVPPVDGYVMVLQPEEPKISLSGVHHFARAASEFESSEGVFLFPELRIISTITREVEPEGDGAEDPTVQESLVSEEIVHDLDTCEVTVEGEELNHEQESLEVDMARLQQKGIEVSSSELGMTFTGVDTMASYEEVLHLLRYRNWHARSLLDRKFKLICSELNGRYISNEFKVEVNVIHTANPMEHANHMAAQPQFVHPEHRSFVDLSGHNLANPHPFAVVPSTATVVIVVCVSFLVFMIILGVFRIRAAHRRTMRDQDTGKENEMDWDDSALTITVNPMETYEDQHSSEEEEEEEEEEESEDGEEEDDITSAESESSEEEEGEQGDPQNATRQQQLEWDDSTLSY

SEQ ID NO: 15 - protein, 350 aa, Homo sapiens [sequence omitted]

SEQ ID NO: 16 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 17 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 18 - protein, 461 aa, Homo sapiens
MPPSGPRGTLLLLPLLLLLLLRAVLAVPLERGAPNKEETPATESPDTGLYYHRYLQEVIDVLETDGHFREKLQAANAEDIKSGKLSRELDFVSHHVRTRLDELKRQEVSRLRMLLKAKMDAEQDPNVQVDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWETVEMHPAYTEEELRRFEEELAAREAELNAKAQRLSQETEALGRSQGRLEAQKRELQQAVLHMEQRKQQQQQQQGHKAPAAHPEGQLKFHPDTDDVPVPAPAGDQKEVDTSEKKLLERLPEVEVPQHL

SEQ ID NO: 19 - protein, 381 aa, Homo sapiens
MSSRIARALALVVTLLHLTRLALSTCPAACHCPLEAPKCAPGVGLVRDGCGCCKVCAKQLNEDCSKTQPCDHTKGLECNFGASSTALKGICRAQSEGRPCEYNSRIYQNGESFQPSCKHQCTCIDGAVGCIPLCPQELSLPNLGCPNPRLVKVTGQCCEEWVCDEDSIKDPMEDQDGLLGKELGFDASEVELTRNNELIAVGKGSSLKRLPVFGMEPRILYNPLQGQKCIVQTTSWSQCSKTCGTGISTRVTNDNPECRLVKETRICEVRPCGQPVYSSLKKGKKCSKTKKSPEPVRFTYAGCLSVKKYRPKYCGSRVDGRCCTPQLTRTVKMRFRCEDGETFSKNVMMIQSCKCNYNCPHANEAAFPFYRLFNDIHKFRG

SEQ ID NO: 20 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 21 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKERYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 22 - protein, 895 aa, Homo sapiens
MRMSVGLSLLLPLWGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 23 - protein, 1479 aa, Homo sapiens
MAKRSRGPGRRCLLALVLFCAWGTLAVVAQKPGAGCPSRCLCFRTTVRCMHLLLEAVPAVAPQTSILDLRFNRIREIQPGAFRRLRNLNTLLLNNNQIKRIPSGAFEDLENLKYLYLYKNEIQSIDRQAFKGLASLEQLYLHFNQIETLDPDSFQHLPKLERLFLHNNRITHLVPGTFNHLESMKRLRLDSNTLHCDCEILWLADLLKTYAESGNAQAAAICEYPRRIQGRSVATITPEELNCERPRITSEPQDADVTSGNTVYFTCRAEGNPKPEIIWLRNNNELSMKTDSRLNLLDDGTLMIQNTQETDQGIYQCMAKNVAGEVKTQEVTLRYFGSPARPTFVIQPQNTEVLVGESVTLECSATGHPPPRISWTRGDRTPLPVDPRVNITPSGGLYIQNVVQGDSGEYACSATNNIDSVHATAFIIVQALPQFTVTPQDRVVIEGQTVDFQCEAKGNPPPVIAWTKGGSQLSVDRRHLVLSSGTLRISGVALHDQGQYECQAVNIIGSQKVVAHLTVQPRVTPVFASIPSDTTVEVGANVQLPCSSQGEPEPAITWNKDGVQVTESGKFHISPEGFLTINDVGPADAGRYECVARNTIGSASVSMVLSVNVPDVSRNGDPFVATSIVEAIATVDRAINSTRTHLFDSRPRSPNDLLALFRYPRDPYTVEQARAGEIFERTLQLIQEHVQHGLMVDLNGTSYHYNDLVSPQYLNLIANLSGCTAHRRVNNCSDMCFHQKYRTHDGTCNNLQHPMWGASLTAFERLLKSVYENGFNTPRGINPHRLYNGHALPMPRLVSTTLIGTETVTPDEQFTHMLMQWGQFLDHDLDSTVVALSQARFSDGQHCSNVCSNDPPCFSVMIPPNDSRARSGARCMFFVRSSPVCGSGMTSLLMNSVYPREQINQLTSYIDASNVYGSTEHEARSIRDLASHRGLLRQGIVQRSGKPLLPFATGPPTECMRDENESPIPCFLAGDHRANEQLGLTSMHTLWFREHNRIATELLKLNPHWDGDTIYYETRKIVGAEIQHITYQHWLPKILGEVGMRTLGEYHGYDPGINAGIFNAFATAAFRFGHTLVNPLLYRLDENFQPIAQDHLPLHKAFFSPFRIVNEGGIDPLLRGLFGVAGKMRVPSQLLNTELTERLFSMAHTVALDLAAINIQRGRDHGIPPYHDYRVYCNLSAAHTFEDLKNEIKNPEIREKLKRLYGSTLNIDLFPALVVEDLVPGSRLGPTLMCLLSTQFKRLRDGDRLWYENPGVFSPAQLTQIKQTSLARILCDNADNITRVQSDVFRVAEFPHGYGSCDEIPRVDLRVWQDCCEDCRTRGQFNAFSYHFRGRRSLEFSYQEDKPTKKTRPRKIPSVGRQGEHLSNSTSAFSTRSDASGTNDFREFVLEMQKTITDLRTQIKKLESRLSTTECVDAGGESHANNTKWKKDACTICECKDGQVTCFVEACPPATCAVPVNIPGACCPVCLQKRAEEKP

SEQ ID NO: 24 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 25 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 26 - protein, 149 aa, Homo sapiens [sequence omitted]

SEQ ID NO: 27 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 28 - protein, 2444 aa, Homo sapiens [sequence omitted]

SEQ ID NO: 29 - protein, 128 aa, Homo sapiens
MQIFVKTLTGKTITLEVEPSDTIENVKAKIQDKEGIPPDQQRLIFAGKQLEDGRTLSDYNIQKESTLHLVLRLRGGIIEPSLRQLAQKYNCDKMICRKCYARLHPRAVNCRKKKCGHTNNLRPKKKVK

SEQ ID NO: 30 - protein, 4391 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS

SEQ ID NO: 31 - protein, 3410 aa, Homo sapiens
IFPPNCNKPPSKAKMFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVLVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 32 - protein, 318 aa, Homo sapiens
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 33 - protein, 774 aa, Homo sapiens
MERPLCSHLCSCLAMLALLSPLSLAQYDSWPHYPEYFQQPAPEYHQPQAPANVAKIQLRLAGQKRKHSEGRVEVYYDGQWGTVCDDDFSIHAAHVVCRELGYVEAKSWTASSSYGKGEGPIWLDNLHCTGNEATLAACTSNGWGVTDCKHTEDVGVVCSDKRIPGFKFDNSLINQIENLNIQVEDIRIRAILSTYRKRTPVMEGYVEVKEGKTWKQICDKHWTAKNSRVVCGMFGFPGERTYNTKVYKMFASRRKQRYWPFSMDCTGTEAHISSCKLGPQVSLDPMKNVTCENGLPAVVSCVPGQVFSPDGPSRFRKAYKPEQPLVRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACSETAPDLVLNAEMVQQTTYLEDRPMFMLQCAMEENCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITMGCWDMYRHDIDCQWVDITDVPPGDYLFQVVINPNFEVAESDYSNNIMKCRSRYDGHRIWMYNCHIGGSFSEETEKKFEHFSGLLNNQLSPQ

SEQ ID NO: 34 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 35 - protein, 353 aa, Homo sapiens
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 36 - protein, 94 aa, Homo sapiens
MEHLEGVINKPEAEMSPQELQLHYFKMHDYDGNNLLDGLELSTAITHVHKEEGSEQAPLMSEDELINIIDGVLRDDDKNNDGYIDYAEFAKSLQ

SEQ ID NO: 37 - protein, 151 aa, Homo sapiens; identical to SEQ ID NO: 4

SEQ ID NO: 38 - protein, 255 aa, Homo sapiens; identical to SEQ ID NO: 5

SEQ ID NO: 39 - protein, 115 aa, Homo sapiens
MRYVASYLLAALGGNSSPSAKDIKKILDSVGIEADDDRLNKVISELNGKNIEDVIAQGIGKLASVPAGGAVAVSAAPGSAAPAAGSAPAAAEEKKDEKKEESEESDDDMGFGLFD

SEQ ID NO: 40 - protein, 2045 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 41 - protein, 892 aa, Homo sapiens
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRFAIQDISVEETSAKEGLLLWCQRKTAPYKNVNIQNFHISWKDGLGFCALIHRHRPELIDYGKLRKDDPLTNLNTAFDVAEKYLDIPKMLDAEDIVGTARPDEKAIMTYVSSFYHAFSGAQKAETAANRICKVLAVNQENEQLMEDYEKLASDLLEWIRRTIPWLENRVPENTMHAMQQKLEDFRDYRRLHKPPKVQEKCQLEINFNTLQTKLRLSNRPAFMPSEGRMVSDINNAWGCLEQVEKGYEEWLLNEIRRLERLDHLAEKFRQKASIHEAWTDGKEAMLRQKDYETATLSEIKALLKKHEAFESDLAAHQDRVEQIAAIAQELNELDYYDSPSVNARCQKICDQWDNLGALTQKRREALERTEKLLETIDQLYLEYAKRAAPFNNWMEGAMEDLQDTFIVHTIEEIQGLTTAHEQFKATLPDADKERLAILGIHNEVSKIVQTYHVNMAGTNPYTTITPQEINGKWDHVRQLVPRRDQALTEEHARQQHNERLRKQFGAQANVIGPWIQTKMEEIGRISIEMHGTLEDQLSHLRQYEKSIVNYKPKIDQLEGDHQLIQEALIFDNKHTNYTMEHIRVGWEQLLTTIARTINEVENQILTRDAKGISQEQMNEFRASFNHFDRDHSGTLGPEEFKACLISLGYDIGNDPQGEAEFARIMSIVDPNRLGVVTFQAFIDFMSRETADTDTADQVMASFKILAGDKNYITMDELRRELPPDQAEYCIARMAPYTGPDSVPGALDYMSFSTALYGESDL

SEQ ID NO: 42 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 43 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 44 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 45 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 46 - protein, 406 aa, Homo sapiens
LGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKGDDEDKDEDEEDEEDKEEDEEEDVPGQAKDEL

SEQ ID NO: 47 - protein, 463 aa, Homo sapiens [sequence omitted]

SEQ ID NO: 48 - protein, 303 aa, Homo sapiens [sequence omitted]

SEQ ID NO: 49 - protein, 603 aa, Homo sapiens
MVKLAKAGKNQGDPKKMAPPPKEVEEDSEDEEMSEDEEDDEDDDDDEEDDSEEEAMETTPAKGKKAAKVVPVKAKGAKNGKNAKKEDSDEEEDDDSEEDEEDDEDEDEDEDEIEPAAMKAAAAAPASEDEDDEDDEDDEDDDDDEDDEDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGNSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 50 - protein, 97 aa, Homo sapiens
AGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILG

SEQ ID NO: 51 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 52 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 53 - protein, 1028 aa, Homo sapiens
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGSSGPSGDEGQPGEPGPPGEKGEAGDEGNPGPDGAPGERGGPGERGPRGTPGTRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVAYGESHLFRVPSYQALLRGVFHQTVSRKVALG

SEQ ID NO: 54 - protein, 3062 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 55 - protein, 3063 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 56 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 57 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 58 - protein, 493 aa, Homo sapiens; identical to SEQ ID NO: 57

SEQ ID NO: 59 - protein, 308 aa, Homo sapiens
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 60 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 61 - protein, 558 aa, Homo sapiens
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 62 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 63 - protein, 854 aa, Homo sapiens
MPPCSGGDGSTPPGPSLRDRDCPAQSAEYPRDRLDPRPGSPSEASSPPFLRSRAPVNWYQEKAQVFLWHLLVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGRELHINLIPNKQGRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 64 - protein, 363 aa, Homo sapiens
SCARGKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 65 - protein, 240 aa, Homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 66 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 67 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 68 - protein, 469 aa, Homo sapiens [sequence omitted]

SEQ ID NO: 69 - protein, 405 aa, Homo sapiens
KQMQEFFGLKVTGKPDAETLKVMKQPRCGVPDVAQFVLTEGNPRWEQTHLTYRIENYTPDLPRADVDHVIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQNDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 70 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 71 - protein, 292 aa, Homo sapiens
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRTMANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 72 - protein, 273 aa, Homo sapiens
MRLPGVPLARPALLLLLPLLAPLLGTGAPAELRVRVRLPDGQVTEESLQADSDADSISLELRKPDGTLVSFTADFKKDVKVFRALILGELEKGQSQFQALCFVTQLQHNEIIPSEAMAKLRQKNPRAVRQAEEVRGLEHLHMDVAVNFSQGALLSPHLHNVCAEAVDAIYTRQEDVRFWLEQGVDSSVFEALPKASEQAELPRCRQVGDHGKPCVCRYGLSLAWYPCMLKYCHSRDRPTPYKCGIRSCQKSYSFDFYVPQRQLCLWDEDPYPG

SEQ ID NO: 73 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 74 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 75 - protein, 343 aa, Homo sapiens
GADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 76 - protein, 179 aa, Homo sapiens
MSGGRAPAVLLGGVASLLLSFVWMPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 77 - protein, 227 aa, Homo sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 78 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 79 - protein, 156 aa, Homo sapiens
MQIFVKTLTGKTITLEVEPSDTIENVKAKIQDKEGIPPDQQRLIFAGKQLEDGRTLSDYNIQKESTLHLVLRLRGGAKKRKKKSYTTPKKNKHKRKKVKLAVLKYYKVDENGKISRLRRECPSDECGAGVFMASHFDRHYCGKCCLTYCFNKPEDK

SEQ ID NO: 80 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 81 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 82 - protein, 323 aa, Homo sapiens [sequence omitted]

SEQ ID NO: 83 - protein, 747 aa, Homo sapiens
MRRCNSGSGPPPSLLLLLLWLLAVPGANAAPRSALYSPSDPLTLLQADTVRGAVLGSRSAWAVEFFASWCGHCIAFAPTWKALAEDVKAWRPALYLAALDCAEETNSAVCRDFNIPGFPTVRFFKAFTKNGSGAVFPVAGADVQTLRERLIDALESHHDTWPPACPPLEPAKLEEIDGFFARNNEEYLALIFEKGGSYLGREVALDLSQHKGVAVRRVLNTEANVVRKFGVTDFPSCYLLFRNGSVSRVPVLMESRSFYTAYLQRLSGLTREAAQTTVAPTTANKIAPTVWKLADRSKIYMADLESALHYILRIEVGRFPVLEGQRLVALKKFVAVLAKYFPGRPLVQNFLHSVNEWLKRQKRNKIPYSFFKTALDDRKEGAVLAKKVNWIGCQGSEPHFRGFPCSLWVLFHFLTVQAARQNVDHSQEAAKAKEVLPAIRGYVHYFFGCRDCASHFEQMAAASMHRVGSPNAAVLWLWSSHNRVNARLAGAPSEDPQFPKVQWPPRELCSACHNERLDVPVWDVEATLNFLKAHFSPSNIILDFPAAGSAARRDVQNVAAAPELAMGALELESRNSTLDPGKPEMMKSPTNTTPHVPAEGPEASRPPKLHPGLRAAPGQEPPEHMAELQRNEQEQPLGQWHLSKRDTGAALLAESRAEKNRLWGPLEVRRVGRSSKQLVDIPEGQLEARAGRGRGQWLQVLGGGFSYLDISLCVGLYSLSFMGLLAMYTYFQAKIRALKGHAGHPAA

SEQ ID NO: 84 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 85 - protein, 589 aa, Homo sapiens
GVQGWRENLCEEREGASREFKGRCEAIMDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 86 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGGERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 87 - protein, 623 aa, Homo sapiens
MESYHKPDQQKLQALKDTANRLRISSIQATTAAGSGHPTSCCSAAEIMAVLFFHTMRYKSQDPRNPHNDRFVLSKGHAAPILYAVWAEAGFLAEAELLNLRKISSDLDGHPVPKQAFTDVATGSLGQGLGAACGMAYTGKYFDKASYRVYCLLGDGELSEGSVWEAMAFASIYKLDNLVAILDINRLGQSDPAPLQHQMDIYQKRCEAFGWHAIIVDGHSVEELCKAFGQAKHQPTAIIAKTFKGRGITGVEDKESWHGKPLPKNMAEQIIQEIYSQIQSKKKILATPPQEDAPSVDIANIRMPSLPSYKVGDKIATRKAYGQALAKLGHASDRIIALDGDTKNSTFSEIFKKEHPDRFIECYIAERNMVSIAVGCATRNRTVPFCSTFAAFFTRAFDQIRMAAISESNINLCGSHCGVSIGEDGPSQMALEDLAMFRSVPTSTVFYPSDGVATEKAVELAANTKGICFIRTSRPENAIIYNNNEDFQVGQAKVVLKSKDDQVTVIGAGVTLHEALAAAELLKKEKINIRVLDPFTIKPLDRKLILDSARATKGRILTVEDHYYEGGIGEAVSSAVVGEPGITVTHLAVNRVPRSGKPAELLKMFGIDRDAIAQAVRGLITKA

SEQ ID NO: 88 - protein, 398 aa, Homo sapiens [sequence omitted]

SEQ ID NO: 89 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 90 - protein, 409 aa, Homo sapiens
MQVTLKTLQQQTFKIDIDPEETVKALKEKIESEKGKDAFPVAGQKLIYAGKILNDDTALKEYKIDEKNFVVVMVTKPKAVSTPAPATTQQSAPASTTAVTSSTTTTVAQAPTPVPALAPTSTPASITPASATASSEPAPASAAKQEKPAEKPAETPVATSPTATDSTSGDSSRSNLFEDATSALVTGQSYENMVTEIMSMGYEREQVIAALRASFNNPDRAVEYLLMGIPGDRESQAVVDPPQAASTGAPQSSAVAAAAATTTATTTTTSSGGHPLEFLRNQPQFQQMRQIIQQNPSLLPALLQQIGRENPQLLQQISQHQEHFIQMLNEPVQEAGGQGGGGGGGSGGIAEAGSGHMNYIQVTPQEKEAIERLKALGFPEGLVIQAYFACEKNENLAANFLLQQNFDED

SEQ ID NO: 91 - protein, 470 aa, Homo sapiens
MMAAMATARVRMGPRCAQALWRMPWLPVFLSLAAAAAAAAAEQQVPLVLWSSDRDLWAPAADTHEGHITSDLQLSTYLDPALELGPRNVLLFLQDKLSIEDFTAYGGVFGNKQDSAFSNLENALDLAPSSLVLPAVDWYAVSTLTTYLQEKLGASPLHVDLATLRELKLNASLPALLLIRLPYTASSGLMAPREVLTGNDEVIGQVLSTLKSEDVPYTAALTAVRPSRVARDVAVVAGGLGRQLLQKQPVSPVIHPPVSYNDTAPRILFWAQNFSVAYKDQWEDLTPLTFGVQELNLTGSFWNDSFARLSLTYERLFGTTVTFKFILANRLYPVSARHWFTMERLEVHSNGSVAYFNASQVTGPSIYSFHCEYVSSLSKKGSLLVARTQPSPWQMMLQDFQIQAFNVMGEQFSYASDCASFFSPGIWMGLLTSLFMLFIFTYGLHMILSLKTMDRFDDHKGPTISLTQIV

SEQ ID NO: 92 - protein, 2768 aa, Homo sapiens
MALVLEIFTLLASICWVSANIFEYQVDAQPLRPCELQRETAFLKQADYVPQCAEDGSFQTVQCQNDGRSCWCVGANGSEVLGSRQPGRPVACLSFCQLQKQQILLSGYINSTDTSYLPQCQDSGDYAPVQCDVQQVQCWCVDAEGMEVYGTRQLGRPKRCPRSCEIRNRRLLHGVGDKSPPQCSAEGEFMPVQCKFVNTTDMMIFDLVHSYNRFPDAFVTFSSFQRRFPEVSGYCHCADSQGRELAETGLELLLDEIYDTIFAGLDLPSTFTETTLYRILQRRFLAVQSVISGRFRCPTKCEVERFTATSFGHPYVPSCRRNGDYQAVQCQTEGPCWCVDAQGKEMHGTRQQGEPPSCAEGQSCASERQQALSRLYFGTSGYFSQHDLFSSPEKRWASPRVARFATSCPPTIKELFVDSGLLRPMVEGQSQQFSVSENLLKEAIRAIFPSRGLARLALQFTTNPKRLQQNLFGGKFLVNVGQFNLSGALGTRGTFNFSQFFQQLGLASFLNGGRQEDLAKPLSVGLDSNSSTGTPEAAKKDGTMNKPTVGSFGFEINLQENQNALKFLASLLELPEFLLFLQHAISVPEDVARDLGDVMETVLSSQTCEQTPERLFVPSCTTEGSYEDVQCFSGECWCVNSWGKELPGSRVRGGQPRCPTDCEKQRARMQSLMGSQPAGSTLFVPACTSEGHFLPVQCFNSECYCVDAEGQAIPGTRSAIGKPKKCPTPCQLQSEQAFLRTVQALLSNSSMLPTLSDTYIPQCSTDGQWRQVQCNGPPEQVFELYQRWEAQNKGQDLTPAKLLVKIMSYREAASGNFSLFIQSLYEAGQQDVFPVLSQYPSLQDVPLAALEGKRPQPRENILLEPYLFWQILNGQLSQYPGSYSDFSTPLAHFDLRNCWCVDEAGQELEGMRSEPSKLPTCPGSCEEAKLRVLQFIRETEEIVSASNSSRFPLGESFLVAKGIRLRNEDLGLPPLFPPREAFAEQFLRGSDYAIRLAAQSTLSFYQRRRFSPDDSAGASALLRSGPYMPQCDAFGSWEPVQCHAGTGHCWCVDEKGGFIPGSLTARSLQIPQCPTTCEKSRTSGLLSSWKQARSQENPSPKDLFVPACLETGEYARLQASGAGTWCVDPASGEELRPGSSSSAQCPSLCNVLKSGVLSRRVSPGYVPACRAEDGGFSPVQCDQAQGSCWCVMDSGEEVPGTRVTGGQPACESPRCPLPFNASEVVGGTILCETISGPTGSAMQQCQLLCRQGSWSVFPPGPLICSLESGRWESQLPQPRACQRPQLWQTIQTQGHFQLQLPPGKMCSADYADLLQTFQVFILDELTARGFCQIQVKTFGTLVSIPVCNNSSVQVGCLTRERLGVNVTWKSRLEDIPVASLPDLHDIERALVGKDLLGRFTDLIQSGSFQLHLDSKTFPAETIRFLQGDHFGTSPRTWFGCSEGFYQVLTSEASQDGLGCVKCPEGSYSQDEECIPCPVGFYQEQAGSLACVPCPVGRTTISAGAFSQTHCVTDCQRNEAGLQCDQNGQYRASQKDRGSGKAFCVDGEGRRLPWWETEAPLEDSQCLMMQKFEKVPESKVIFDANAPVAVRSKVPDSEFPVMQCLTDCTEDEACSFFTVSTTEPEISCDFYAWTSDNVACMTSDQKRDALGNSKATSFGSLRCQVKVRSHGQDSPAVYLKKGQGSTTTLQKRFEPTGFQNMLSGLYNPIVFSASGANLTDAHLFCLLACDRDLCCDGFVLTQVQGGAIICGLLSSPSVLLCNVKDWMDPSEAWANATCPGVTYDQESHQVILRLGDQEFIKSLTPLEGTQDTFTNFQQVYLWKDSDMGSRPESMGCRKDTVPRPASPTEAGLTTELFSPVDLNQVIVNGNQSLSSQKHWLFKHLFSAQQANLWCLSRCVQEHSFCQLAEITESASLYFTCTLYPEAQVCDDIMESNAQGCRLILPQMPKALFRKKVILEDKVKNFYTRLPFQKLMGISIRNKVPMSEKSISNGFFECERRCDADPCCTGFGFLNVSQLKGGEVTCLTLNSLGIQMCSEENGGAWRILDCGSPDIEVHTYPFGWYQKPIAQNNAPSFCPLVVLPSLTEKVSLDSWQSLALSSVVVDPSIRHFDVAHVSTAATSNFSAVRDLCLSECSQHEACLITTLQTQPGAVRCMFYADTQSCTHSLQGQNCRLLLREEATHIYRKPGISLLSYEASVPSVPISTHGRLLGRSQAIQVGTSWKQVDQFLGVPYAAPPLAERRFQAPEPLNWTGSWDASKPRASCWQPGTRTSTSPGVSEDCLYLNVFIPQNVAPNASVLVFFHNTMDREESEGWPAIDGSFLAAVGNLIVVTASYRVGVFGFLSSGSGEVSGNWGLLDQVAALTWVQTHIRGFGGDPRRVSLAADRGGADVASIHLLTARATNSQLFRRAVLMGGSALSPAAVISHERAQQQAIALAKEVSCPMSSSQEVVSCLRQKPANVLNDAQTKLLAVSGPFHYWGPVIDGHFLREPPARALKRSLWVEVDLLIGSSQDDGLINRAKAVKQFEESRGRTSSKTAFYQALQNSLGGEDSDARVEAAATWYYSLEHSTDDYASFSRALENATRDYFIICPIIDMASAWAKRARGNVFMYHAPENYGHGSLELLADVQFALGLPFYPAYEGQFSLEEKSLSLKIMQYFSHFIRSGNPNYPYEFSRKVPTFATPWPDFVPRAGGENYKEFSELLPNRQGLKKADCSFWSKYISSLKTSADGAKGGQSAESEEEELTAGSGLREDLLSLQEPGSKTYSK

SEQ ID NO: 93 - protein, 766 aa, Homo sapiens [sequence omitted]

SEQ ID NO: 94 - protein, 431 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRGKHFISL

SEQ ID NO: 95 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD